(19) United States
(12) Patent Application Publication
(10) Pub. No.: US 2024/0374726 A1
(43) Pub. Date: Nov. 14, 2024

(54) BAG6 SPECIFIC CHIMERIC ANTIGEN RECEPTOR, NUCLEIC ACID, BAG6 SPECIFIC CHIMERIC ANTIGEN RECEPTOR EXPRESSION PLASMID, BAG6 SPECIFIC CHIMERIC ANTIGEN RECEPTOR EXPRESSING CELL, USE THEREOF, AND PHARMACEUTICAL COMPOSITION FOR TREATING CANCER

(71) Applicant: China Medical University Hospital, Taichung City (TW)

(72) Inventors: Der-Yang Cho, Taichung City (TW); Shao-Chih Chiu, Taichung City (TW); Chia-Ing Jan, Taichung City (TW); Chih-Ming Pan, Taichung City (TW); Shi-Wei Huang, Taichung City (TW)

(73) Assignee: China Medical University Hospital, Taichung City (TW)

(21) Appl. No.: 18/654,284

(22) Filed: May 3, 2024

(30) Foreign Application Priority Data
May 9, 2023 (TW) 112117199

(51) Int. Cl. A61K 39/00; A61K 31/282; A61K 31/495; A61K 31/704; A61K 31/7068; A61P 35/00; C07K 14/705; C07K 14/725; C07K 16/28; C12N 5/0783

(52) U.S. Cl. CPC A61K 39/464411; A61K 31/282; A61K 31/495; A61K 31/704; A61K 31/7068; A61K 39/4631; A61P 35/00; C07K 14/7051; C07K 14/70517; C07K 14/70521; C07K 14/70578; C07K 16/2827; C07K 16/2833; C12N 5/0636; C12N 5/0646; C07K 2319/03; C07K 2319/30; C12N 2510/00

(57) ABSTRACT

A BAG6 specific chimeric antigen receptor, a nucleic acid, a BAG6 specific chimeric antigen receptor expression plasmid, a BAG6 specific chimeric antigen receptor expressing cell, a pharmaceutical composition for treating cancer, and use of the BAG6 specific chimeric antigen receptor expressing cell are provided. The BAG6 specific chimeric antigen receptor specifically binds to BCL2 associated athanogene 6 (BAG6). The nucleic acid encodes the BAG6 specific chimeric antigen receptor. The BAG6 specific chimeric antigen receptor expression plasmid expresses the BAG6 specific chimeric antigen receptor. The BAG6 specific chimeric antigen receptor expressing cell is obtained by transducing the BAG6 specific chimeric antigen receptor into an immune cell. The pharmaceutical composition for treating cancer includes the BAG6 specific chimeric antigen receptor expressing cell and a pharmaceutically acceptable carrier.

### RELATED APPLICATIONS

This application claims priority to Taiwan Application Serial Number 112117199, filed May 9, 2023, which is herein incorporated by reference.

### SEQUENCE LISTING XML

A sequence listing XML submitted as an xml file via EFS-WEB is incorporated herein by reference. The sequence listing XML file submitted via EFS-WEB with the name “CP-4878-US_SEQ_LIST” was created on Mar. 26, 2024, which is 31,500 bytes in size.

### BACKGROUND

### Technical Field

The present disclosure relates to a pharmaceutical product including an antigen or an antibody. More particularly, the present disclosure relates to a chimeric antigen receptor, a nucleic acid encoding the chimeric antigen receptor, a chimeric antigen receptor expression plasmid, a chimeric antigen receptor expressing cell, a pharmaceutical composition for treating cancer, and use of the chimeric antigen receptor expressing cell.

### Description of Related Art

Cancer, also known as malignancy, is a state of abnormal proliferation of cells, and these proliferating cells may invade other parts of the body as a disease caused by a malfunction in the control of cell division and proliferation. The number of people suffering from cancer worldwide has a growing trend. Cancer is one of the top ten causes of death for the Chinese people and has been the top ten causes of death for twenty-seven consecutive years.

Conventional cancer treatments include surgery, radiation therapy, chemotherapy, and target therapy. Cancer immunotherapy is another method for treating cancer except the above methods. The immune system of the patient is activated in the cancer immunotherapy by using tumor cells or tumor antigens to induce specific cellular and humoral immune responses for enhancing the anti-cancer ability of the patient, preventing the growth, spread, and recurrence of tumors, and achieving the purpose of removing or controlling tumors.

There are three main directions for the cancer immunotherapy: the tumor vaccine, the cell therapy and the immune checkpoint inhibitor. The chimeric antigen receptor immune cell technology is one of the cell therapy developing very rapidly in recent years. In conventional technology, the chimeric antigen receptor immune cell transfects a chimeric protein, which couples the antigen binding portion having capable of recognizing a certain tumor antigen of the antibody to the intracellular portion of the CD3-δ chain or FcεRIγ in vitro, into the immune cell by a transduction method to express the chimeric antigen receptor. The chimeric antigen receptor immune cell technology has a significant therapeutic effect in the treatment of acute leukemia and non-Hodgkin's lymphoma, and it is considered to be one of the most promising treatments for cancer. However, the cell therapy of the chimeric antigen receptor immune cell currently has the following disadvantages: lack of unique tumor-associated antigens, low efficiency of homing of immune cells to tumor sites, and inability to overcome the immunosuppressive microenvironment of solid tumors. Accordingly, the efficacy of the chimeric antigen receptor immune cell in solid tumors is greatly limited.

### SUMMARY

According to one aspect of the present disclosure, a BAG6 specific chimeric antigen receptor specific to BCL2 associated athanogene 6 (BAG6) includes, in order from an N-terminus to a C-terminus, a BAG6 antigen recognition domain, a transmembrane domain and a cytoplasmic domain. The BAG6 antigen recognition domain includes a monoclonal antibody fragment specific to BAG6, and the BAG6 antigen recognition domain includes the amino acid sequence of SEQ ID NO: 1.

According to another aspect of the present disclosure, a nucleic acid encoding the aforementioned BAG6 specific chimeric antigen receptor includes, in order from a 5′ end to a 3′ end, a BAG6 antigen recognition domain coding fragment including the nucleic acid sequence of SEQ ID NO: 13, a transmembrane domain coding fragment and a cytoplasmic domain coding fragment.

According to still another aspect of the present disclosure, a BAG6 specific chimeric antigen receptor expression plasmid includes, in order from a 5′ end to a 3′ end, a promoter including the nucleic acid sequence of SEQ ID NO: 25 and the aforementioned nucleic acid.

According to yet another aspect of the present disclosure, a BAG6 specific chimeric antigen receptor expressing cell includes an immune cell and the aforementioned BAG6 specific chimeric antigen receptor expression plasmid.

According to further another aspect of the present disclosure, a pharmaceutical composition for treating cancer includes the aforementioned BAG6 specific chimeric antigen receptor expressing cell and a pharmaceutically acceptable carrier.

According to still another aspect of the present disclosure, a method for inhibiting a proliferation of a tumor cell includes administering a composition including a plurality of the aforementioned BAG6 specific chimeric antigen receptor expressing cells to a subject in need for a treatment of a tumor.

### BRIEF DESCRIPTION OF THE DRAWINGS

The present disclosure can be more fully understood by reading the following detailed description of the embodiment, with reference made to the accompanying drawings as follows:

FIG. 1 is a schematic view showing a construction of a BAG6 specific chimeric antigen receptor according to one aspect of the present disclosure.

FIG. 2A shows analytical results of immunofluorescence staining assay of the expression level of BAG6 of the tumor cells treated with a chemotherapy drug.

FIG. 2B shows analytical results of the flow cytometry of the expression level of BAG6 of the tumor cells treated with the chemotherapy drug.

FIG. 3 shows analytical results of the flow cytometry of the expression level of HLA-G of the tumor cells treated with the chemotherapy drug.

FIG. 4 shows analytical results of the flow cytometry of the expression level of B7-H6 of the tumor cells treated with the chemotherapy drug.

FIG. 5 is a schematic view showing a construction of a BAG6 specific chimeric antigen receptor expression plasmid according to 1st embodiment of another aspect of the present disclosure.

FIG. 6 is a schematic view showing the theoretical structure and mechanism of a BAG6 specific chimeric antigen receptor according to 1st embodiment of one aspect of the present disclosure.

FIG. 7A, FIG. 7B, FIG. 7C, FIG. 7D, FIG. 7E, FIG. 7F, FIG. 7G and FIG. 7H show analytical results of tumor cell death induced by the BAG6 specific chimeric antigen receptor expressing cells according to Example 1 of the present disclosure.

FIG. 8A, FIG. 8B, FIG. 8C, FIG. 8D, FIG. 8E, FIG. 8F, FIG. 8G and FIG. 8H show analytical results of tumor cell death induced by the BAG6 specific chimeric antigen receptor expressing cells according to Example 2 of the present disclosure.

FIG. 9 is a schematic view showing a construction of a BAG6 specific chimeric antigen receptor expression plasmid according to 2nd embodiment of another aspect of the present disclosure.

FIG. 10 is a schematic view showing the theoretical structure and mechanism of a BAG6 specific chimeric antigen receptor according to 2nd embodiment of one aspect of the present disclosure.

FIG. 11A, FIG. 11B, FIG. 11C, FIG. 11D, FIG. 11E, FIG. 11F, FIG. 11G and FIG. 11H show analytical results of tumor cell death induced by the BAG6 specific chimeric antigen receptor expressing cells according to Example 3 of the present disclosure.

FIG. 12A, FIG. 12B, FIG. 12C, FIG. 12D, FIG. 12E, FIG. 12F, FIG. 12G and FIG. 12H show analytical results of tumor cell death induced by the BAG6 specific chimeric antigen receptor expressing cells according to Example 4 of the present disclosure.

FIG. 13 is a schematic view showing a construction of a BAG6 specific chimeric antigen receptor expression plasmid according to 3rd embodiment of another aspect of the present disclosure.

FIG. 14 is a schematic view showing the theoretical structure and mechanism of a BAG6 specific chimeric antigen receptor according to 3rd embodiment of one aspect of the present disclosure.

FIG. 15A, FIG. 15B, FIG. 15C, FIG. 15D, FIG. 15E, FIG. 15F, FIG. 15G and FIG. 15H show analytical results of tumor cell death induced by the BAG6 specific chimeric antigen receptor expressing cells according to Example 5 of the present disclosure.

FIG. 16A, FIG. 16B, FIG. 16C, FIG. 16D, FIG. 16E, FIG. 16F, FIG. 16G and FIG. 16H show analytical results of tumor cell death induced by the BAG6 specific chimeric antigen receptor expressing cells according to Example 6 of the present disclosure.

### DETAILED DESCRIPTION

A BAG6 specific chimeric antigen receptor, a nucleic acid encoding the BAG6 specific chimeric antigen receptor, a BAG6 specific chimeric antigen receptor expression plasmid including the nucleic acid, a BAG6 specific chimeric antigen receptor expressing cell including the BAG6 specific chimeric antigen receptor expression plasmid, a use thereof, and a pharmaceutical composition for treating cancer including the BAG6 specific chimeric antigen receptor expressing cell are provided.

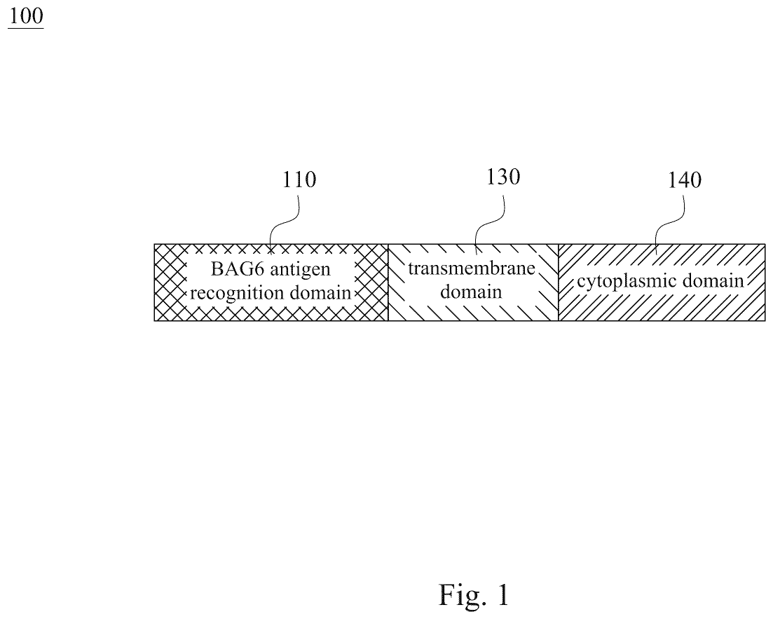

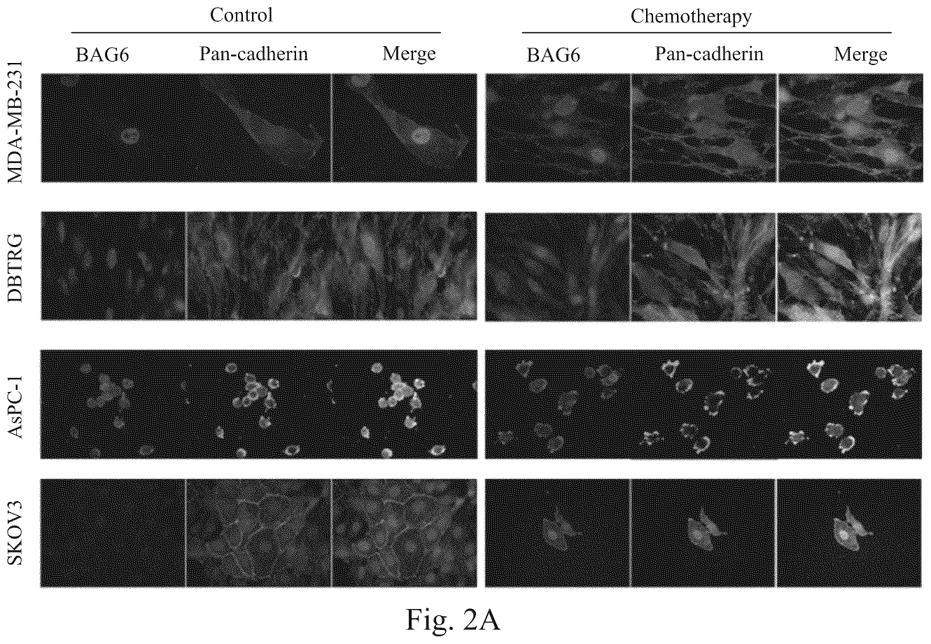

Reference is made to FIG. 1, which is a schematic view showing a construction of a BAG6 specific chimeric antigen receptor 100 according to one aspect of the present disclosure. The BAG6 specific chimeric antigen receptor 100 specifically binds to BCL2 associated athanogene 6 (BAG6). The BAG6 specific chimeric antigen receptor 100 includes, in order from an N-terminus to a C-terminus, a BAG6 antigen recognition domain 110, a transmembrane domain 130 and a cytoplasmic domain 140. The BAG6 antigen recognition domain 110 includes a monoclonal antibody fragment specific to BAG6, and the BAG6 antigen recognition domain 110 includes the amino acid sequence of SEQ ID NO: 1. The BAG6 specific chimeric antigen receptor 100 can further include a HLA-G antigen recognition domain including the amino acid sequence of SEQ ID NO: 2, wherein the HLA-G antigen recognition domain is linked to the C-terminus of the BAG6 antigen recognition domain 110, and the HLA-G antigen recognition domain includes a monoclonal antibody fragment specific to human leukocyte antigen G (HLA-G). In addition, the BAG6 specific chimeric antigen receptor 100 can further include a B7-H6 antigen recognition domain including the amino acid sequence of SEQ ID NO: 3, wherein the B7-H6 antigen recognition domain is linked to the C-terminus of the BAG6 antigen recognition domain 110, and the B7-H6 antigen recognition domain includes a monoclonal antibody fragment specific to B7 homolog 6 (B7-H6).

The transmembrane domain 130 of the BAG6 specific chimeric antigen receptor 100 can be a CD28 transmembrane domain including the amino acid sequence of SEQ ID NO: 4, a CD8 transmembrane domain including the amino acid sequence of SEQ ID NO: 5 or a KIR transmembrane domain including the amino acid sequence of SEQ ID NO: 6. The cytoplasmic domain 140 can be a IL2 receptor β chain signaling domain including the amino acid sequence of SEQ ID NO: 7, a modified CD3ζ signaling domain including the amino acid sequence of SEQ ID NO: 8, a DAP12 signaling domain including the amino acid sequence of SEQ ID NO: 9, a CD3ζ signaling domain including the amino acid sequence of SEQ ID NO: 10, a 4-1BB signaling domain including the amino acid sequence of SEQ ID NO: 11 or a combination thereof. The BAG6 specific chimeric antigen receptor 100 can further include a suicide protein including the amino acid sequence of SEQ ID NO: 12, wherein the suicide protein is linked to the C-terminus of the cytoplasmic domain 140.

According to another aspect of the present disclosure, a nucleic acid encodes the aforementioned BAG6 specific chimeric antigen receptor 100. The nucleic acid includes, in order from a 5′ end to a 3′ end, a BAG6 antigen recognition domain coding fragment including the nucleic acid sequence of SEQ ID NO: 13, a transmembrane domain coding fragment and a cytoplasmic domain coding fragment. The nucleic acid can further include a HLA-G antigen recognition domain coding fragment including the nucleic acid sequence of SEQ ID NO: 14, wherein the HLA-G antigen recognition domain coding fragment is linked to the 3′ end of the BAG6 antigen recognition domain coding fragment. In addition, the nucleic acid can further include a B7-H6 antigen recognition domain coding fragment including the nucleic acid sequence of SEQ ID NO: 15, wherein the B7-H6 antigen recognition domain is linked to the 3′ end of the BAG6 antigen recognition domain coding fragment.

The transmembrane domain coding fragment can be a CD28 transmembrane domain coding fragment including the nucleic acid sequence of SEQ ID NO: 16, a CD8 transmembrane domain coding fragment including the nucleic acid sequence of SEQ ID NO: 17 or a KIR transmembrane domain coding fragment including the nucleic acid sequence of SEQ ID NO: 18. The cytoplasmic domain coding fragment can be a IL2 receptor β chain signaling domain coding fragment including the nucleic acid sequence of SEQ ID NO: 19, a modified CD3ζ signaling domain coding fragment including the nucleic acid sequence of SEQ ID NO: 20, a DAP12 signaling domain coding fragment including the nucleic acid sequence of SEQ ID NO: 21, a CD3ζ signaling domain coding fragment including the nucleic acid sequence of SEQ ID NO: 22, a 4-1BB signaling domain coding fragment including the nucleic acid sequence of SEQ ID NO: 23 or a combination thereof. In addition, the nucleic acid can further include the nucleic acid sequence of SEQ ID NO: 24, wherein the suicide gene is linked to the 3′ end of the cytoplasmic domain coding fragment.

According to still another aspect of the present disclosure, a BAG6 specific chimeric antigen receptor expression plasmid includes, in order from a 5′ end to a 3′ end, a promoter including the nucleic acid sequence of SEQ ID NO: 25 and the aforementioned nucleic acid. In addition, the BAG6 specific chimeric antigen receptor expression plasmid can further include a suicide gene including the nucleic acid sequence of SEQ ID NO: 24, wherein the suicide gene is linked to the 3′ end of the nucleic acid.

According to yet another aspect of the present disclosure, a BAG6 specific chimeric antigen receptor expressing cell includes an immune cell and the aforementioned BAG6 specific chimeric antigen receptor expression plasmid. The immune cell can be a T lymphocyte or a natural killer (NK) cell.

According to further another aspect of the present disclosure, a pharmaceutical composition for treating cancer includes the aforementioned BAG6 specific chimeric antigen receptor expressing cell and a pharmaceutically acceptable carrier. The pharmaceutical composition for treating cancer can further include a chemotherapy drug. Preferably, the chemotherapy drug can be doxorubicin, temozolomide, gemcitabine or carboplatin.

According to still another aspect of the present disclosure, a method for inhibiting a proliferation of a tumor cell includes administering a composition including a plurality of the aforementioned BAG6 specific chimeric antigen receptor expressing cells to a subject in need for a treatment of a tumor. The tumor cell can be a breast cancer cell, a glioblastoma multiforme cell, a pancreatic cancer cell or an ovarian cancer cell.

A tumor cell specific binding ability of the BAG6 specific chimeric antigen receptor of the present disclosure, especially a specific binding ability to BAG6 expressed on the cell membrane of tumor cells, is confirmed by in vitro cell assay of the tumor cells. Accordingly, the BAG6 specific chimeric antigen receptor expressing cell of the present disclosure, which expresses the BAG6 specific chimeric antigen receptor of the present disclosure, can specifically target the tumor cells to avoid the off-target effect, thereby effectively killing the tumor cells. Therefore, the BAG6 specific chimeric antigen receptor expressing cell can be used for inhibiting the proliferation of the tumor cells in the subject in need for the treatment of the tumor. The pharmaceutical composition for treating cancer of the present disclosure includes the BAG6 specific chimeric antigen receptor expressing cell of the present disclosure, and can further include a chemotherapy drug, which can effectively kill tumor cells and thereby treat cancer.

Reference will now be made in detail to the present embodiments of the present disclosure, examples of which are illustrated in the accompanying drawings.

### I. Treatment With the Chemotherapy Drug Increases the Expression of BAG6, HLA-G and B7-H6 on the Cell Membrane of Tumor Cells

To explore whether treatment with the chemotherapy drug affects the expression of BAG6, HLA-G and B7-H6 on the cell membrane of tumor cells, the tumor cells used were human breast cancer cell line MDA-MB-231, human malignant brain tumor cell line DBTRG-05MG (hereinafter referred to as DBTRG), human pancreatic cancer cell line AsPC-1, and human ovarian cancer cell line SKOV3. The tumor cells used were all purchased from the American Type Culture Collection (ATCC). The human breast cancer cell line MDA-MB-231 is a triple-negative breast cancer cell line, that is, the hormone receptor (ER, PR) and HER-2 receptor thereof are negative, and the human breast cancer cell line MDA-MB-231 was cultured in RPMI culture medium including 10% fetal bovine serum (FBS). The human malignant brain tumor cell line DBTRG was cultured in DMEM culture medium including 10% FBS. The human pancreatic cancer cell line AsPC-1 was cultured in RPMI culture medium including 10% FBS. The human ovarian cancer cell line SKOV3 was cultured in McCoy's 5A culture medium including 10% FBS.

First, the human breast cancer cell line MDA-MB-231, the human malignant brain tumor cell line DBTRG, the human pancreatic cancer cell line AsPC-1 and the human ovarian cancer cell line SKOV3 were seeded in a 6-well plate at a density of 2×10⁵ cells/well. The cells were subsequently incubated for 24 hours. Each type of the tumor cells was divided into two groups. In a control, the tumor cells were untreated. In a chemotherapy group, the tumor cells were treated with the chemotherapy drug for 48 hours. The chemotherapy drug used for treating the human breast cancer cell line MDA-MB-231 was doxorubicin (200 nM), the chemotherapy drug used for treating the human malignant brain tumor cell line DBTRG was temozolomide (80 μg/mL), the chemotherapy drug used for treating the human pancreatic cancer cell line AsPC-1 was gemcitabine (20 μM), and the chemotherapy drug used for treating the human ovarian cancer cell line SKOV3 was carboplatin (20 μM). Then, the expression of BAG6 of the tumor cells of each group were detected by immunofluorescence staining assay, and the expression of BAG6, HLA-G and B7-H6 of the tumor cells of each group were detected by the flow cytometry.

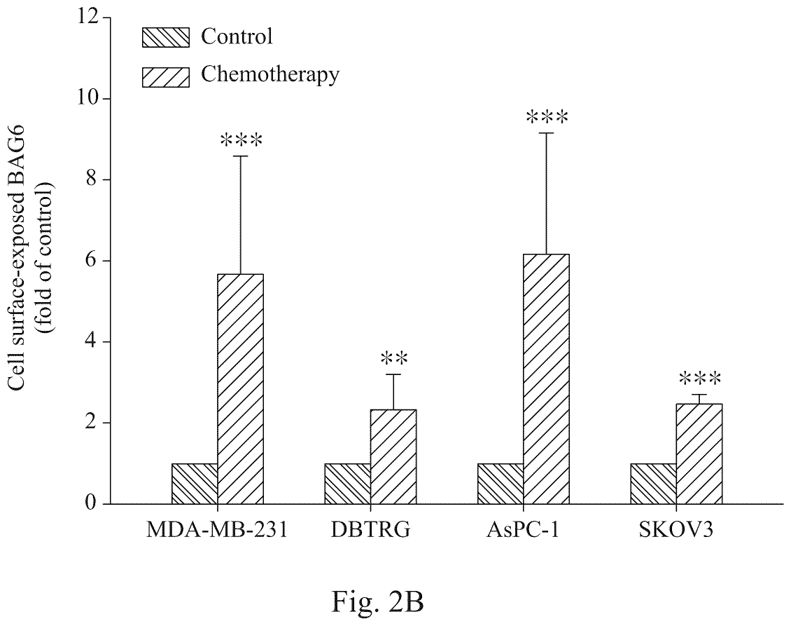

Reference is made to FIG. 2A and FIG. 2B. FIG. 2A shows analytical results of immunofluorescence staining assay of the expression level of BAG6 of the tumor cells treated with the chemotherapy drug, and FIG. 2B shows analytical results of the flow cytometry of the expression level of BAG6 of the tumor cells treated with the chemotherapy drug.

As shown by the results of FIG. 2A and FIG. 2B, treatment of doxorubicin could increase the expression of BAG6 on the plasma membrane of the human breast cancer cell line MDA-MB-231, and there was a statistically significant difference between the control and the chemotherapy group (p<0.001). Treatment of temozolomide could increase the expression of BAG6 on the plasma membrane of the human malignant brain tumor cell line DBTRG, and there was a statistically significant difference between the control and the chemotherapy group (p<0.01). Treatment of gemcitabine could increase the expression of BAG6 on the plasma membrane of the human pancreatic cancer cell line AsPC-1, and there was a statistically significant difference between the control and the chemotherapy group (p<0.001). Treatment of carboplatin could increase the expression of BAG6 on the plasma membrane of the human ovarian cancer cell line SKOV3, and there was a statistically significant difference between the control and the chemotherapy group (p<0.001).

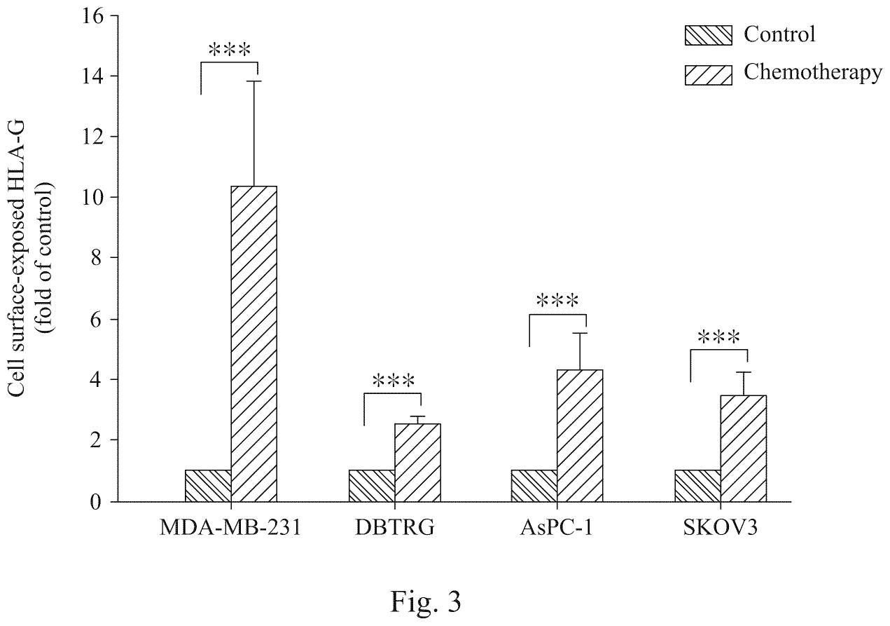

Reference is made to FIG. 3, which shows analytical results of the flow cytometry of the expression level of HLA-G of the tumor cells treated with the chemotherapy drug. The results show that treatment of doxorubicin could increase the expression of HLA-G on the plasma membrane of the human breast cancer cell line MDA-MB-231, and there was a statistically significant difference between the control and the chemotherapy group (p<0.001). Treatment of temozolomide could increase the expression of HLA-G on the plasma membrane of the human malignant brain tumor cell line DBTRG, and there was a statistically significant difference between the control and the chemotherapy group (p<0.001). Treatment of gemcitabine could increase the expression of HLA-G on the plasma membrane of the human pancreatic cancer cell line AsPC-1, and there was a statistically significant difference between the control and the chemotherapy group (p<0.001). Treatment of carboplatin could increase the expression of HLA-G on the plasma membrane of the human ovarian cancer cell line SKOV3, and there was a statistically significant difference between the control and the chemotherapy group (p<0.001).

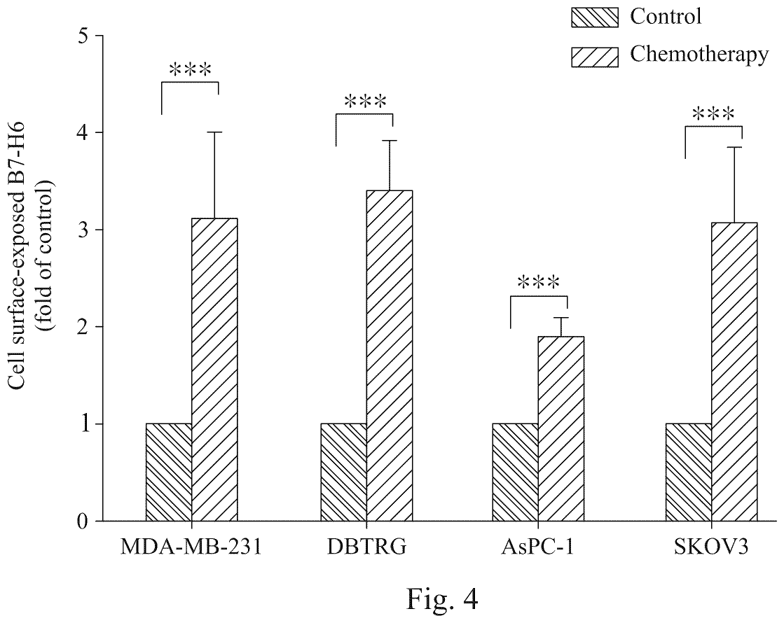

Reference is made to FIG. 4, which shows analytical results of the flow cytometry of the expression level of B7-H6 of the tumor cells treated with the chemotherapy drug. The results show that treatment of doxorubicin could increase the expression of B7-H6 on the plasma membrane of the human breast cancer cell line MDA-MB-231, and there was a statistically significant difference between the control and the chemotherapy group (p<0.001). Treatment of temozolomide could increase the expression of B7-H6 on the plasma membrane of the human malignant brain tumor cell line DBTRG, and there was a statistically significant difference between the control and the chemotherapy group (p<0.001). Treatment of gemcitabine could increase the expression of B7-H6 on the plasma membrane of the human pancreatic cancer cell line AsPC1, and there was a statistically significant difference between the control and the chemotherapy group (p<0.001). Treatment of carboplatin could increase the expression of B7-H6 on the plasma membrane of the human ovarian cancer cell line SKOV3, and there was a statistically significant difference between the control and the chemotherapy group (p<0.001).

### II. 1st Embodiment

A BAG6 specific chimeric antigen receptor of 1st embodiment includes, in order from an N-terminus to a C-terminus, the BAG6 antigen recognition domain including the amino acid sequence of SEQ ID NO: 1, the CD28 transmembrane domain including the amino acid sequence of SEQ ID NO: 4, the IL2 receptor β chain signaling domain including the amino acid sequence of SEQ ID NO: 7, and the modified CD3ζ signaling domain including the amino acid sequence of SEQ ID NO: 8. A nucleic acid of 1st embodiment includes, in order from a 5′ end to a 3′ end, the BAG6 antigen recognition domain coding fragment including the nucleic acid sequence of SEQ ID NO: 13, the CD28 transmembrane domain coding fragment including the nucleic acid sequence of SEQ ID NO: 16, the IL2 receptor β chain signaling domain coding fragment including the nucleic acid sequence of SEQ ID NO: 19, and the modified CD3ζ signaling domain coding fragment including the nucleic acid sequence of SEQ ID NO: 20.

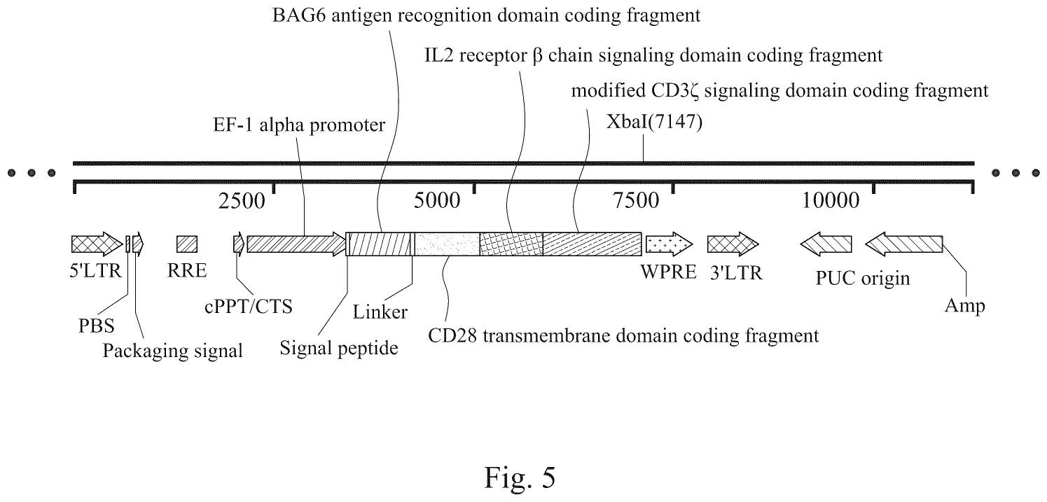

Reference is made to FIG. 5, which is a schematic view showing a construction of a BAG6 specific chimeric antigen receptor expression plasmid according to 1st embodiment of another aspect of the present disclosure. In detail, the insert of the BAG6 specific chimeric antigen receptor expression plasmid of 1st embodiment includes in order from a 5′ end to a 3′ end, the promoter including the nucleic acid sequence of SEQ ID NO: 25, the BAG6 antigen recognition domain coding fragment including the nucleic acid sequence of SEQ ID NO: 13, the CD28 transmembrane domain coding fragment including the nucleic acid sequence of SEQ ID NO: 16, and the cytoplasmic domain coding fragment. The cytoplasmic domain coding fragment includes the IL2 receptor β chain signaling domain coding fragment including the nucleic acid sequence of SEQ ID NO: 19, and the modified CD3ζ signaling domain coding fragment including the nucleic acid sequence of SEQ ID NO: 20. Then, the insert is constructed on Creative Biolabs vector (Creative Biolabs, NY, USA) to obtain the BAG6 specific chimeric antigen receptor expression plasmid of 1st embodiment. The Creative Biolabs vector is a lentivirus vector system, so that the constructed BAG6 specific chimeric antigen receptor expression plasmid of 1st embodiment can be transfected into expression cells to produce lentiviruses, and the BAG6 specific chimeric antigen receptor expression plasmid of 1st embodiment can be subsequently transduced into the immune cells using lentiviruses.

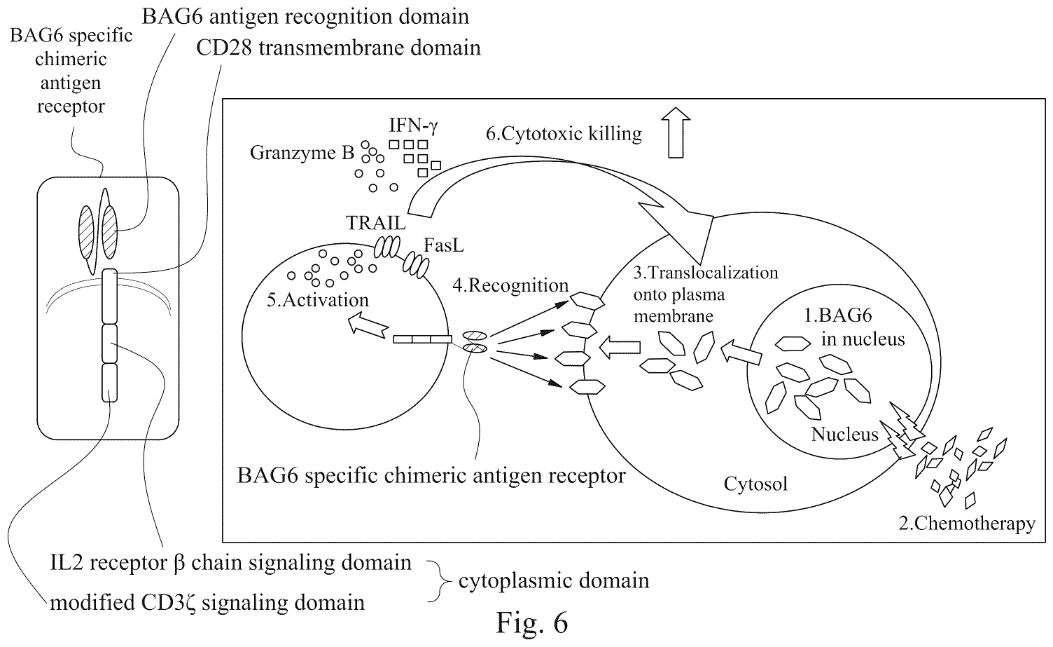

Reference is made to FIG. 6, which is a schematic view showing the theoretical structure and mechanism of the BAG6 specific chimeric antigen receptor according to 1st embodiment of one aspect of the present disclosure. A BAG6 specific chimeric antigen receptor expressing cell of 1st embodiment is a genetically engineered NK cell or T cell that expresses the BAG6 specific chimeric antigen receptor of 1st embodiment, wherein the BAG6 specific chimeric antigen receptor of 1st embodiment is a tumor-targeting receptor complex consisted of the BAG6 antigen recognition domain, the CD28 transmembrane domain and the cytoplasmic domain. The cytoplasmic domain includes the IL2 receptor β chain signaling domain and the modified CD3ζ signaling domain. The BAG6-specific chimeric antigen receptor of 1st embodiment can specifically recognize BAG6 on the tumor cell membrane, thereby killing the tumor cells. Preferably, when the tumor cells are treated with the chemotherapy drug, BAG6 in the nucleus can be translocated onto the plasma membrane of the tumor cells. The BAG6-specific chimeric antigen receptor of 1st embodiment binds to BAG6 specifically recognized on the surface of the tumor cells and triggers signal transduction, resulting in a signal cascade leading to the activation and the proliferation of the BAG6 specific chimeric antigen receptor expressing cell of the present disclosure, thereby triggering exocytosis of lytic granules and killing the target tumor cells.

### 2.1. Example 1

The BAG6 specific chimeric antigen receptor of 1st embodiment was transduced into the primary T lymphocyte to obtain a BAG6 specific chimeric antigen receptor expressing cell of Example 1 of the present disclosure (hereinafter referred to as Example 1). The effects of Example 1 and the pharmaceutical composition for treating cancer including Example 1 of the present disclosure on inducing the death of the breast cancer cells, the glioblastoma multiforme cells, the pancreatic cancer cells, and the ovarian cancer cells were further demonstrated in following experiments.

First, the human breast cancer cell line MDA-MB-231, the human malignant brain tumor cell line DBTRG, the human pancreatic cancer cell line AsPC-1 and the human ovarian cancer cell line SKOV3 were seeded in a 12-well plate at a density of 1×10⁵ cells/well. The cells were subsequently incubated for 48 hours. Each type of the tumor cells was divided into six groups. In a control, the tumor cells were untreated. In a group 1, the tumor cells were treated with the chemotherapy drug. In a group 2, the tumor cells were treated with the parental primary T lymphocyte. In a group 3, the tumor cells are treated with the parental primary T lymphocyte and the chemotherapy drug. In a group 4, the tumor cells were treated with Example 1. In a group 5, the tumor cells were treated with Example 1 and the chemotherapy drug. The chemotherapy drug used for treating the human breast cancer cell line MDA-MB-231 was doxorubicin (200 nM), the chemotherapy drug used for treating the human malignant brain tumor cell line DBTRG was temozolomide (80 μg/mL), the chemotherapy drug used for treating the human pancreatic cancer cell line AsPC-1 was gemcitabine (20 μM), and the chemotherapy drug used for treating the human ovarian cancer cell line SKOV3 was carboplatin (20 μM). In the group 4 and the group 5, the number of Example 1 treated was 1×10⁵ cells. In the group 2 and the group 3, the number of the parental primary T lymphocyte treated was 1×10⁵ cells. The treated cells of each group were stained with Annexin V-FITC and propidium iodide (PI), and the apoptosis and the death of the tumor cells were detected by the flow cytometry. The sum of the percentage of cells stained with Annexin V-FITC and/or PI were calculated to obtain the cytotoxicity. The results of the cytotoxicity were counted after three independent trials in each group.

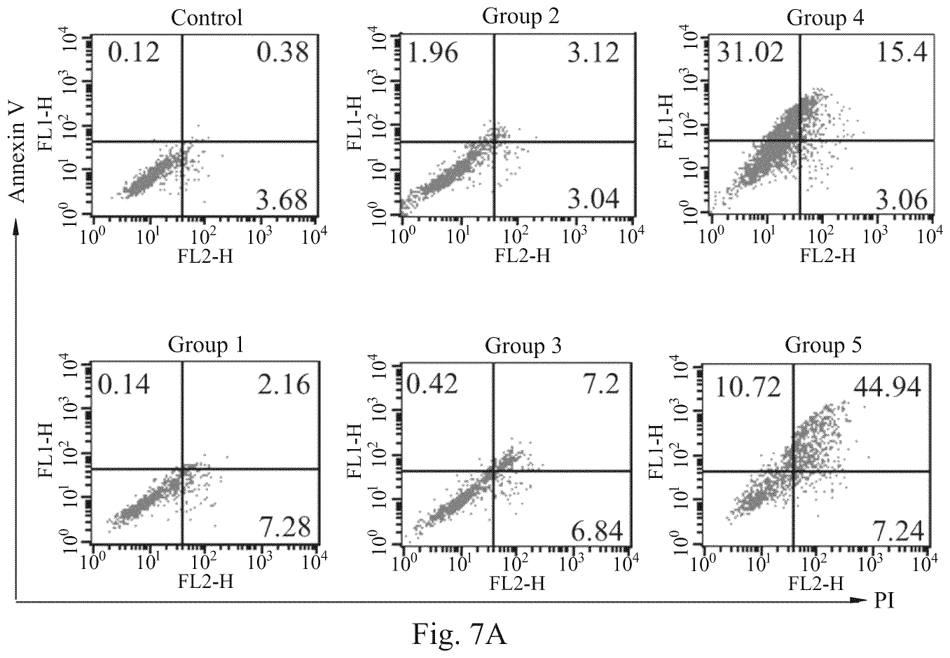

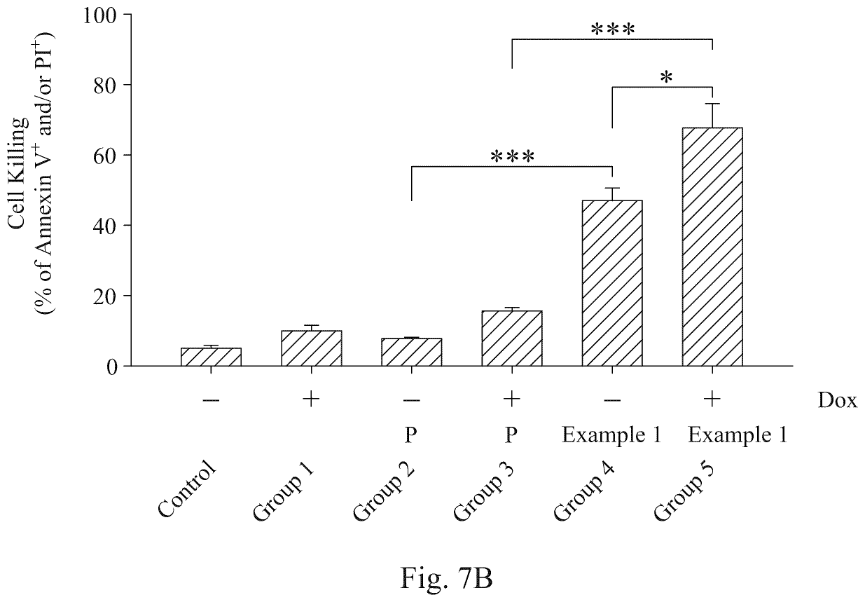

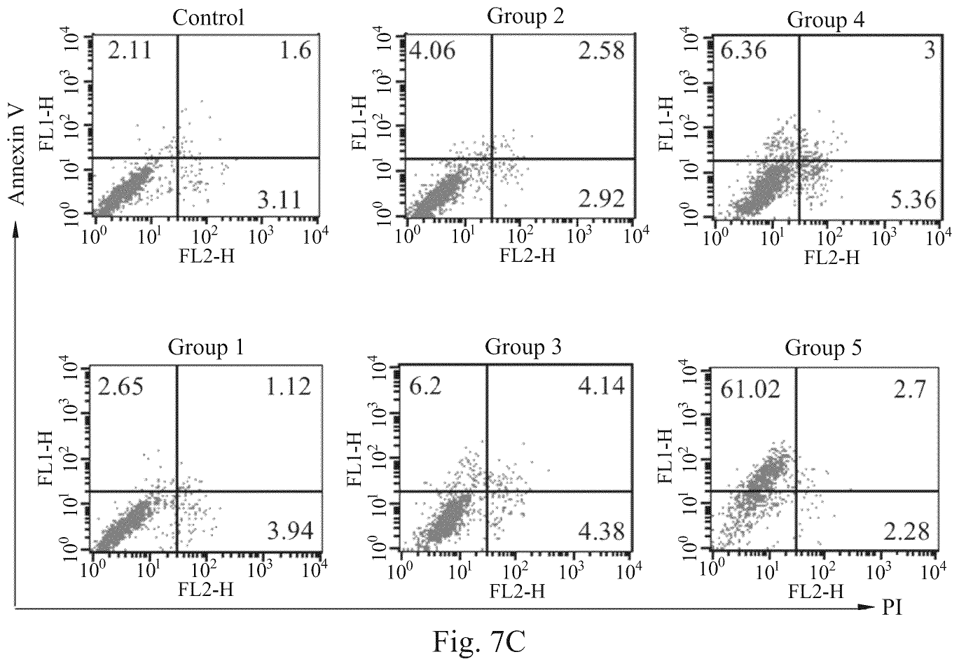

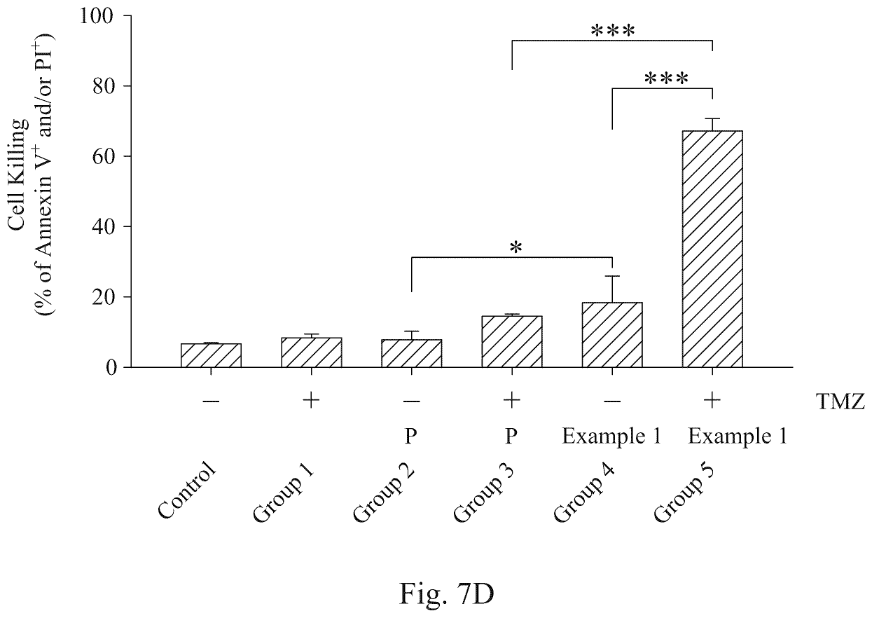

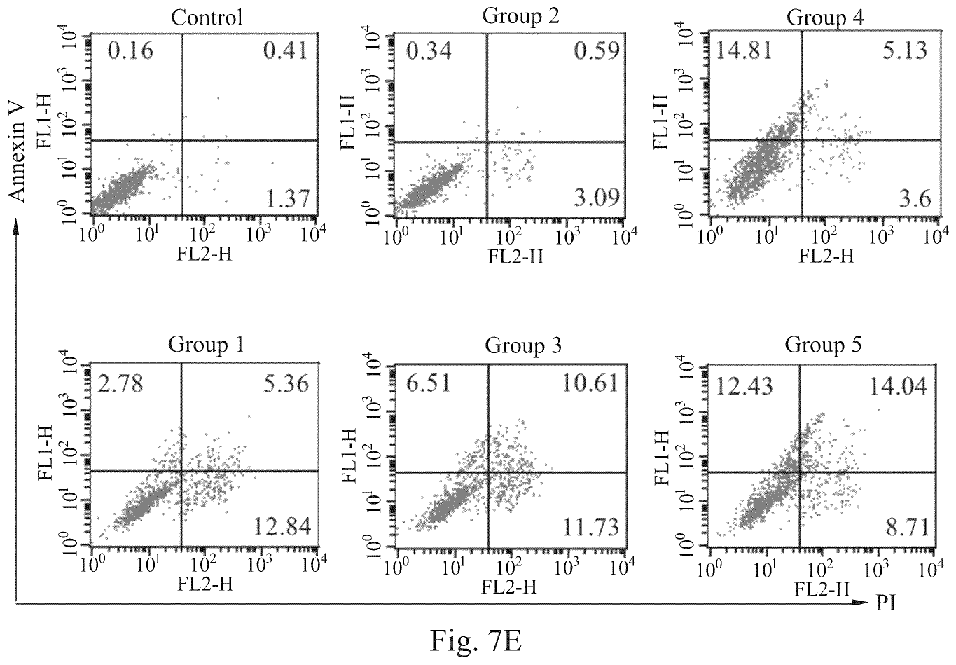

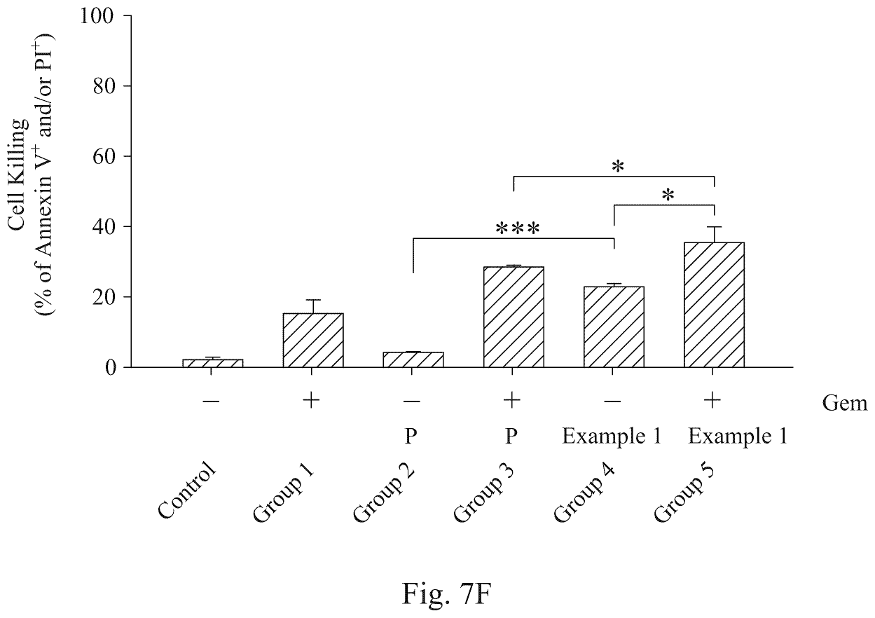

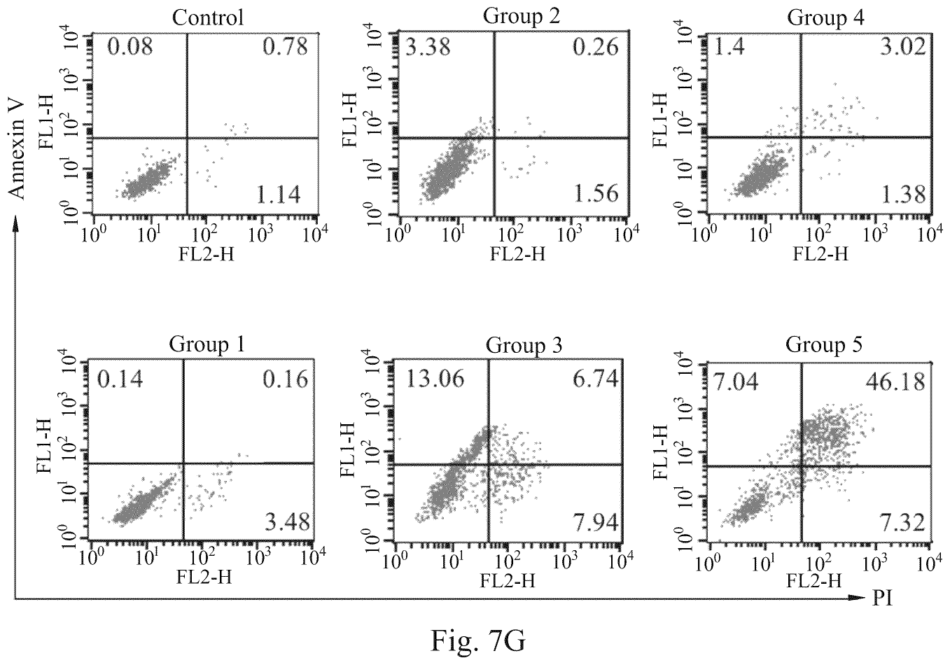

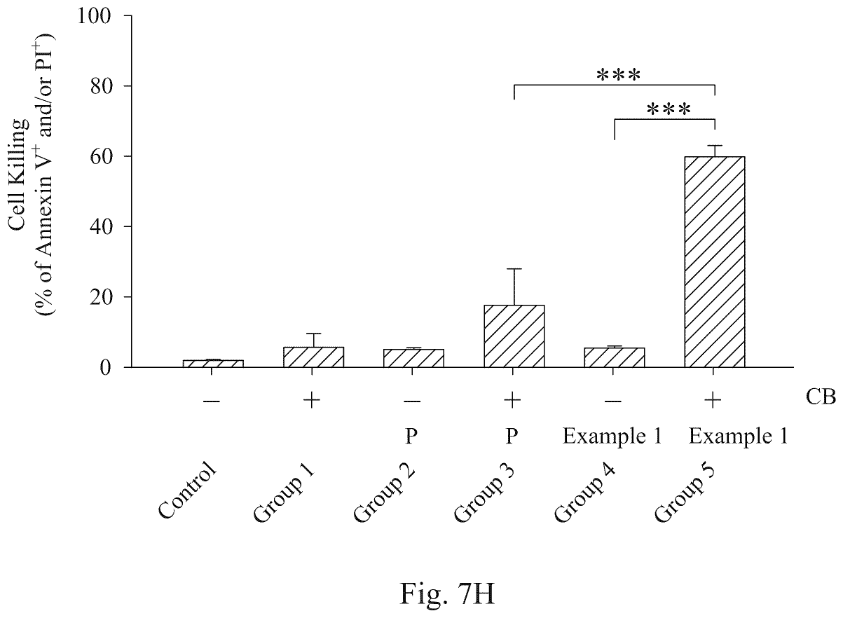

Reference is made to FIG. 7A, FIG. 7B, FIG. 7C, FIG. 7D, FIG. 7E, FIG. 7F, FIG. 7G, FIG. 7H and Table 1, which show analytical results of tumor cell death induced by the BAG6 specific chimeric antigen receptor expressing cells according to Example 1 of the present disclosure, wherein “P” represents parental primary T cells in FIG. 7B, FIG. 7D, FIG. 7F and FIG. 7H. FIG. 7A is a graph showing the analytical results of the human breast cancer cell line MDA-MB-231, and FIG. 7B is a statistical chart of FIG. 7A after three independent trials, wherein “Dox” represents doxorubicin. FIG. 7C is a graph showing the analytical results of the human malignant brain tumor cell line DBTRG, and FIG. 7D is a statistical chart of FIG. 7C after three independent trials, wherein “TMZ” represents temozolomide. FIG. 7E is a graph showing the analytical results of the human pancreatic cancer cell line AsPC-1, and FIG. 7F is a statistical chart of FIG. 7E after three independent trials, wherein “Gem” represents gemcitabine. FIG. 7G is a graph showing the analytical results of the human ovarian cancer cell line SKOV3, and FIG. 7H is a statistical chart of FIG. 7G after three independent trials, wherein “CB” represents carboplatin. Table 1 shows the death rate of each group.

TABLE 1
Analytical results of Example 1

| | Control | Group 1 | Group 2 | Group 3 | Group 4 | Group 5 |
|---|---|---|---|---|---|---|
| MDA- | 5.0800 ± | 10.0067 ± | 7.7733 ± | 15.5867 ± | 47.0200 ± | 67.7300 ± |
| MB-231 | 0.7800 | 1.5837 | 0.3844 | 1.0187 | 3.5355 | 6.8307 |
| DBTRG | 6.6950 ± | 8.4033 ± | 7.8450 ± | 14.5367 ± | 18.3833 ± | 67.1933 ± |
| | 0.1768 | 1.0736 | 2.4254 | 0.6158 | 7.5319 | 3.4883 |
| AsPC-1 | 2.1633 ± | 15.2080 ± | 4.2233 ± | 28.5150 ± | 22.8650 ± | 35.4367 ± |
| | 0.7118 | 3.9265 | 0.1914 | 0.4738 | 0.9546 | 4.4006 |
| SKOV3 | 1.9400 ± | 5.7467 ± | 5.0667 ± | 17.5986 ± | 5.4933 ± | 59.8333 ± |
| | 0.2553 | 3.8655 | 0.4549 | 10.3675 | 0.5672 | 3.2384 |

The results of Table 1, FIG. 7A and FIG. 7B show that in the human breast cancer cell line MDA-MB-231, the death rate of the human breast cancer cell line MDA-MB-231 in the group 4 treated with Example 1 was approximately 47%, and there was a statistically significant difference (p<0.001) compared to the group 2. In addition, the death rate of the human breast cancer cell line MDA-MB-231 in the group 5 treated with Example 1 and doxorubicin was as high as 67.7%, and there was a statistically significant difference (p<0.05) compared to the group 4 and a statistically significant difference (p<0.001) compared to the group 3, respectively.

The results of Table 1, FIG. 7C and FIG. 7D show that in the human malignant brain tumor cell line DBTRG, the death rate of the human malignant brain tumor cell line DBTRG in the group 4 treated with Example 1 was 18.4%, and there was a statistically significant difference (p<0.05) compared to the group 2. In addition, the death rate of the human malignant brain tumor cell line DBTRG in the group 5 treated with Example 1 and temozolomide was close to 67.2%, and there was a statistically significant difference (p<0.001) compared to the group 4 and a statistically significant difference (p<0.001) compared to the group 3, respectively.

The results of Table 1, FIG. 7E and FIG. 7F show that in the human pancreatic cancer cell line AsPC-1, the death rate of the human pancreatic cancer cell line AsPC-1 in the group 4 treated with Example 1 was about 22.9%, and there was a statistically significant difference (p<0.001) compared to the group 2. In addition, the death rate of the human pancreatic cancer cell line AsPC-1 in the group 5 treated with Example 1 and gemcitabine was 35.4%, and there was a statistically significant difference (p<0.05) compared to the group 4 and a statistically significant difference (p<0.05) compared to the group 3, respectively.

The results of Table 1, FIG. 7G and FIG. 7H show that in the human ovarian cancer cell line SKOV3, the death rate of the human ovarian cancer cell line SKOV3 in the group 4 treated with Example 1 was about 5.5%. The death rate of the human ovarian cancer cell line SKOV3 in the group 5 treated with Example 1 and carboplatin could reach 59.8%, and there was a statistically significant difference (p<0.001) compared to the group 4 and a statistically significant difference (p<0.001) compared to the group 3, respectively.

### 2.2. Example 2

The BAG6 specific chimeric antigen receptor of 1st embodiment was transduced into the primary NK cell to obtain a BAG6 specific chimeric antigen receptor expressing cell of Example 2 of the present disclosure (hereinafter referred to as Example 2). The effects of Example 2 and the pharmaceutical composition for treating cancer including Example 2 of the present disclosure on inducing the death of the breast cancer cells, the glioblastoma multiforme cells, the pancreatic cancer cells, and the ovarian cancer cells were further demonstrated in following experiments.

First, the human breast cancer cell line MDA-MB-231, the human malignant brain tumor cell line DBTRG, the human pancreatic cancer cell line AsPC-1 and the human ovarian cancer cell line SKOV3 were seeded in a 12-well plate at a density of 1×10⁵ cells/well. The cells were subsequently incubated for 48 hours. Each type of the tumor cells was divided into six groups. In a control, the tumor cells were untreated. In a group 1, the tumor cells were treated with the chemotherapy drug. In a group 2, the tumor cells were treated with the parental primary NK cell. In a group 3, the tumor cells were treated with the parental primary NK cell and the chemotherapy drug. In a group 4, the tumor cells were treated with Example 2. In a group 5, the tumor cells were treated with Example 2 and the chemotherapy drug. The chemotherapy drug used for treating the human breast cancer cell line MDA-MB-231 was doxorubicin (200 nM), the chemotherapy drug used for treating the human malignant brain tumor cell line DBTRG was temozolomide (80 μg/mL), the chemotherapy drug used for treating the human pancreatic cancer cell line AsPC-1 was gemcitabine (20 μM), and the chemotherapy drug used for treating the human ovarian cancer cell line SKOV3 was carboplatin (20 μM). In the group 4 and the group 5, the number of Example 2 treated was 1×10⁵ cells. In the group 2 and the group 3, the number of the parental primary NK cell treated was 1×10⁵ cells. The treated cells of each group were stained with Annexin V-FITC and PI, and the apoptosis and the death of the tumor cells were detected by the flow cytometry. The sum of the percentage of cells stained with Annexin V-FITC and/or PI were calculated to obtain the cytotoxicity. The results of the cytotoxicity were counted after three independent trials in each group.

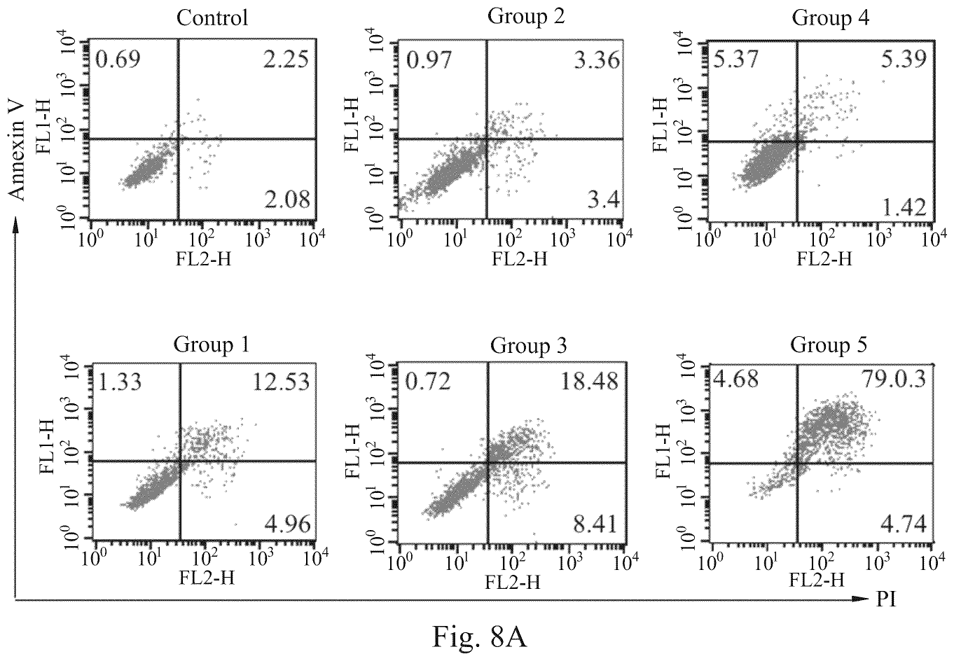

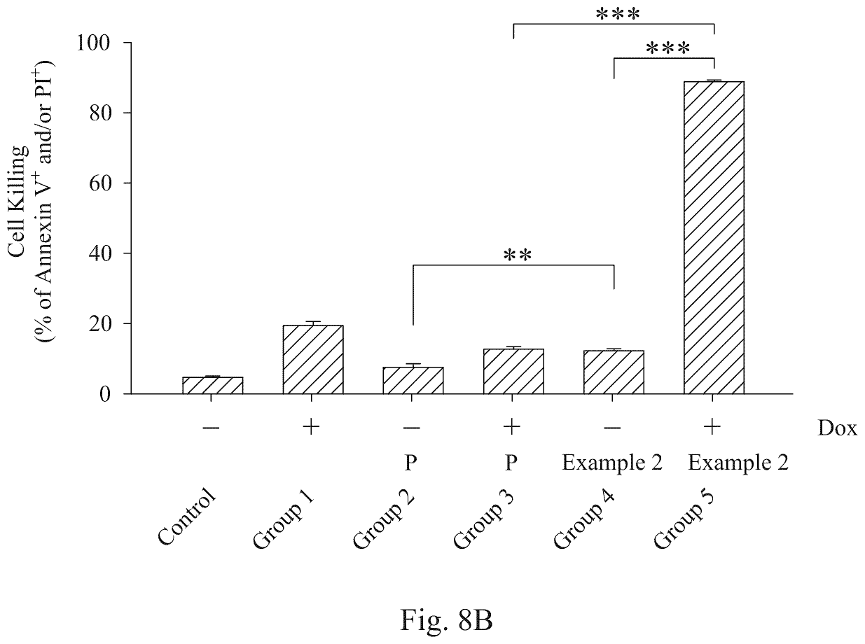

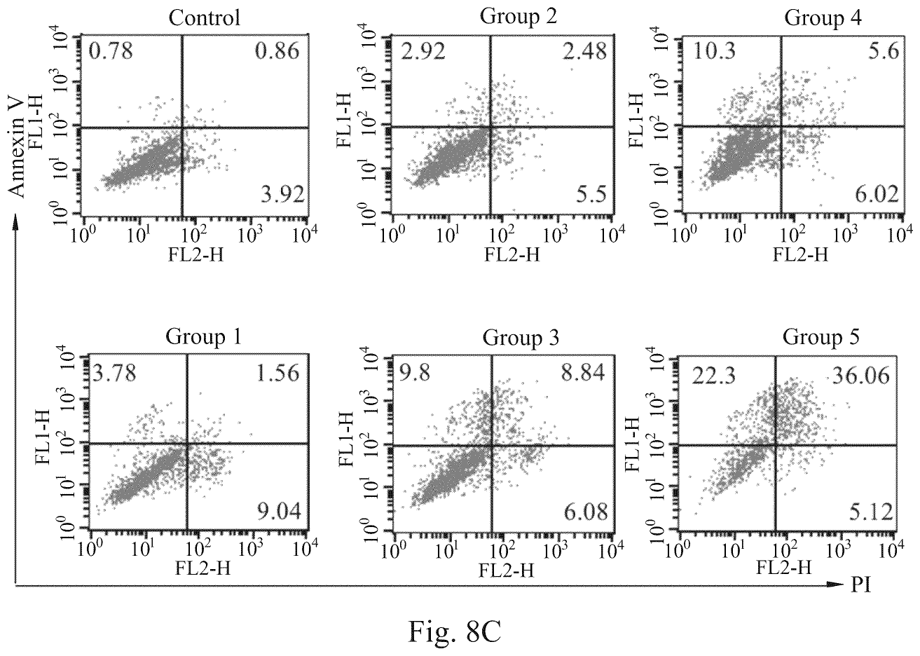

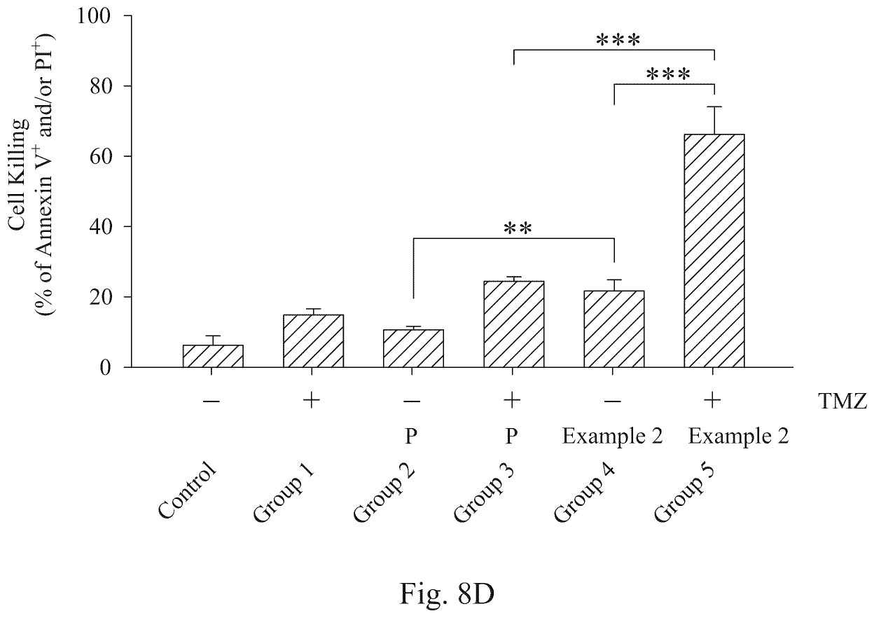

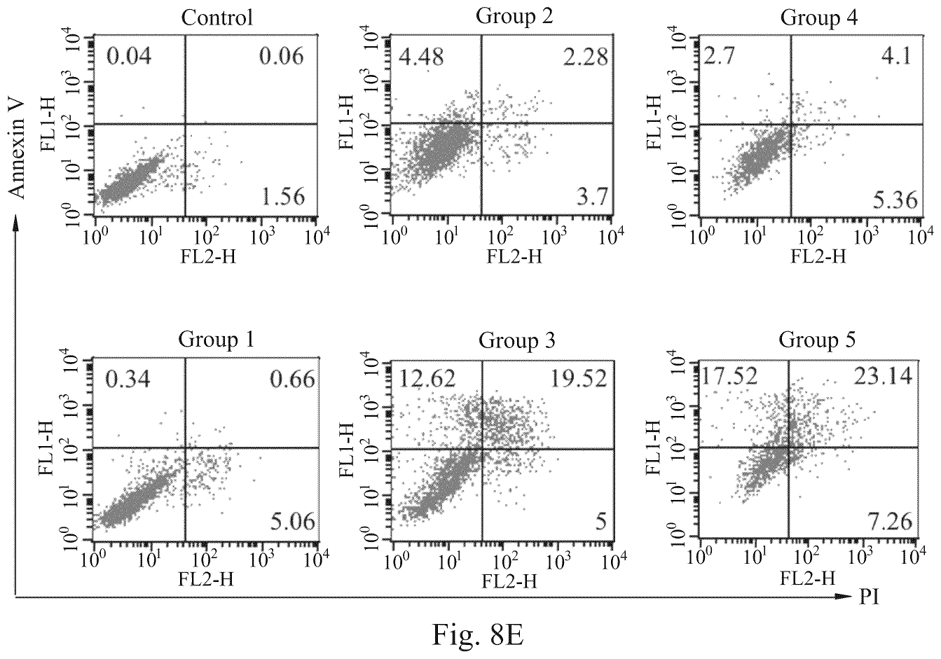

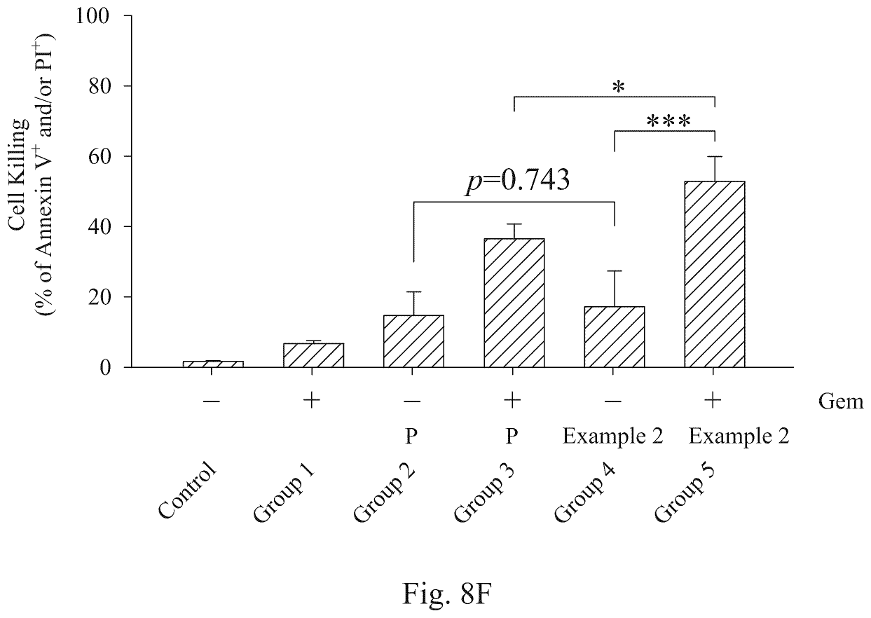

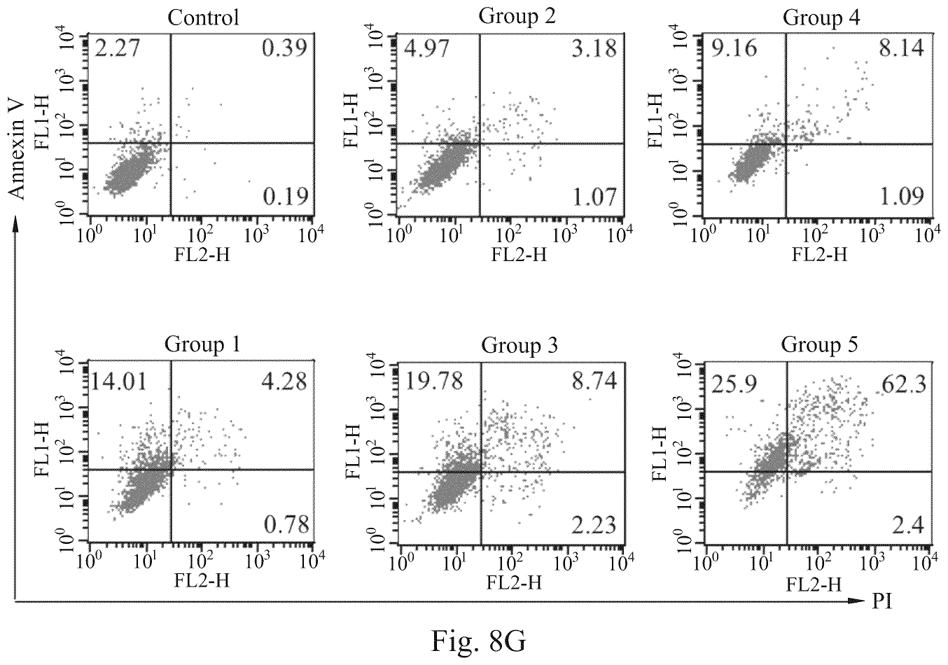

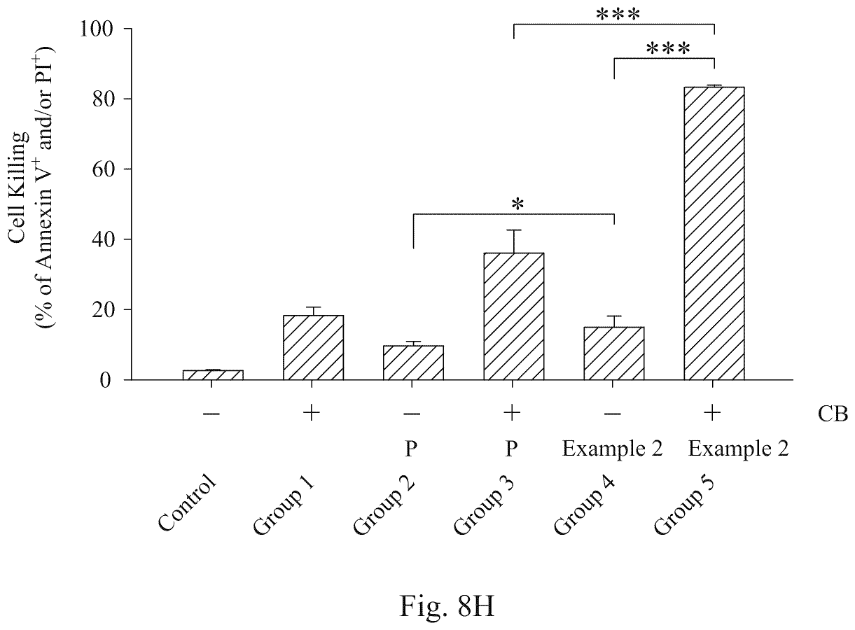

Reference is made to FIG. 8A, FIG. 8B, FIG. 8C, FIG. 8D, FIG. 8E, FIG. 8F, FIG. 8G, FIG. 8H and Table 2, which show analytical results of tumor cell death induced by the BAG6 specific chimeric antigen receptor expressing cells according to Example 2 of the present disclosure, wherein “P” represents parental primary NK cell in FIG. 8B, FIG. 8D, FIG. 8F and FIG. 8H. FIG. 8A is a graph showing the analytical results of the human breast cancer cell line MDA-MB-231, and FIG. 8B is a statistical chart of FIG. 8A after three independent trials, wherein “Dox” represents doxorubicin. FIG. 8C is a graph showing the analytical results of the human malignant brain tumor cell line DBTRG, and FIG. 8D is a statistical chart of FIG. 8C after three independent trials, wherein “TMZ” represents temozolomide. FIG. 8E is a graph showing the analytical results of the human pancreatic cancer cell line AsPC-1, and FIG. 8F is a statistical chart of FIG. 8E after three independent trials, wherein “Gem” represents gemcitabine. FIG. 8G is a graph showing the analytical results of the human ovarian cancer cell line SKOV3, and FIG. 8H is a statistical chart of FIG. 8G after three independent trials, wherein “CB” represents carboplatin. Table 2 shows the death rate of each group.

TABLE 2
Analytical results of Example 2

| | Control | Group 1 | Group 2 | Group 3 | Group 4 | Group 5 |
|---|---|---|---|---|---|---|
| MDA- | 4.7100 ± | 19.4900 ± | 7.5100 ± | 12.6800 ± | 12.2967 ± | 88.7950 ± |
| MB-231 | 0.3568 | 1.0889 | 1.0607 | 0.7495 | 0.4856 | 0.4879 |
| DBTRG | 6.1933 ± | 14.9267 ± | 10.6400 ± | 24.3600 ± | 21.7000 ± | 66.2000 ± |
| | 2.7453 | 1.7070 | 0.8969 | 1.3758 | 3.1358 | 7.8421 |
| AsPC-1 | 1.6933 ± | 6.7267 ± | 14.7067 ± | 36.5200 ± | 17.1667 ± | 52.8900 ± |
| | 0.0416 | 0.8769 | 6.7248 | 4.1846 | 10.2158 | 7.0286 |
| SKOV3 | 2.5800 ± | 18.2533 ± | 9.6400 ± | 35.9867 ± | 14.9433 ± | 83.1900 ± |
| | 0.2488 | 2.4111 | 1.2443 | 6.6072 | 3.1609 | 0.6327 |

The results of Table 2, FIG. 8A and FIG. 8B show that in the human breast cancer cell line MDA-MB-231, the death rate of the human breast cancer cell line MDA-MB-231 in the group 4 treated with Example 2 could reach 12.3%, and there was a statistically significant difference (p<0.01) compared to the group 2. In addition, the death rate of the human breast cancer cell line MDA-MB-231 in the group 5 treated with Example 2 and doxorubicin was over 80%, and there was a statistically significant difference (p<0.001) compared to the group 4 and a statistically significant difference (p<0.001) compared to the group 3, respectively.

The results of Table 2, FIG. 8C and FIG. 8D show that in the human malignant brain tumor cell line DBTRG, the death rate of the human malignant brain tumor cell line DBTRG in the group 4 treated with Example 2 was about 21.7%, and there was a statistically significant difference (p<0.01) compared to the group 2. In addition, the death rate of the human malignant brain tumor cell line DBTRG in the group 5 treated with Example 2 and temozolomide was close to 66.2%, and there was a statistically significant difference (p<0.001) compared to the group 4 and a statistically significant difference (p<0.001) compared to the group 3, respectively.

The results of Table 2, FIG. 8E and FIG. 8F show that in the human pancreatic cancer cell line AsPC-1, the death rate of the human pancreatic cancer cell line AsPC-1 in the group 4 treated with Example 2 was about 17.2%. The death rate of the human pancreatic cancer cell line AsPC-1 in the group 5 treated with Example 2 and gemcitabine could reach 52.9%, and there was a statistically significant difference (p<0.001) compared to the group 4 and a statistically significant difference (p<0.05) compared to the group 3, respectively.

The results of Table 2, FIG. 8G and FIG. 8H show that in the human ovarian cancer cell line SKOV3, the death rate of the human ovarian cancer cell line SKOV3 in the group 4 treated with Example 2 was about 14.9%, and there was a statistically significant difference (p<0.05) compared to the group 2. In addition, the death rate of the human ovarian cancer cell line SKOV3 in the group 5 treated with Example 2 and carboplatin was over 80%, and there was a statistically significant difference (p<0.001) compared to the group 4 and a statistically significant difference (p<0.001) compared to the group 3, respectively.

The results of Table 1, Table 2 and FIG. 7A to FIG. 8H show that the BAG6 specific chimeric antigen receptor expressing cell of 1st embodiment can be used to treat with the breast cancer cell, the glioblastoma multiforme cell, the pancreatic cancer cell or the ovarian cancer cell for excellent cell killing. Therefore, the BAG6 specific chimeric antigen receptor expressing cell of the present disclosure can be used for inhibiting the proliferation of the tumor cells in the subject in need for the treatment of the tumor. In addition, the results of FIG. 2A and FIG. 2B show that treatment with the chemotherapy drug increased the expression of BAG6 on the cell membrane of the tumor cells, and the BAG6 specific chimeric antigen receptor expressed by the BAG6 specific chimeric antigen receptor expressing cell of 1st embodiment can specifically bind to BAG6. Therefore, the treatment of treating the chemotherapy drug first and then treating the BAG6 specific chimeric antigen receptor expressing cell of 1st embodiment, or the simultaneous treatment of the chemotherapy drug and the BAG6 specific chimeric antigen receptor expressing cell of 1st embodiment can achieve a better effect on killing the tumor cells. The results indicate that the pharmaceutical composition for treating cancer of the present disclosure can effectively inhibit the growth of the tumor cells and treat cancer. Preferably, the pharmaceutical composition for treating cancer can include the chemotherapy drug.

### III. 2nd Embodiment

A BAG6 specific chimeric antigen receptor of 2nd embodiment includes, in order from an N-terminus to a C-terminus, the BAG6 antigen recognition domain including the amino acid sequence of SEQ ID NO: 1, the HLA-G antigen recognition domain including the amino acid sequence of SEQ ID NO: 2, the KIR transmembrane domain including the amino acid sequence of SEQ ID NO: 6, and the DAP12 signaling domain including the amino acid sequence of SEQ ID NO: 9. In addition, the BAG6 specific chimeric antigen receptor of 2nd embodiment further includes the suicide protein including the amino acid sequence of SEQ ID NO: 12, which is linked to the C-terminus of the DAP12 signaling domain. A nucleic acid of 2nd embodiment includes, in order from a 5′ end to a 3′ end, the BAG6 antigen recognition domain coding fragment including the nucleic acid sequence of SEQ ID NO: 13, the HLA-G antigen recognition domain coding fragment including the nucleic acid sequence of SEQ ID NO: 14, the KIR transmembrane domain coding fragment including the nucleic acid sequence of SEQ ID NO: 18, and the DAP12 signaling domain coding fragment including the nucleic acid sequence of SEQ ID NO: 21. In addition, the nucleic acid of 2nd embodiment further includes the suicide gene including the nucleic acid sequence of SEQ ID NO: 24, which is linked to the 3′ end of the DAP12 signaling domain coding fragment.

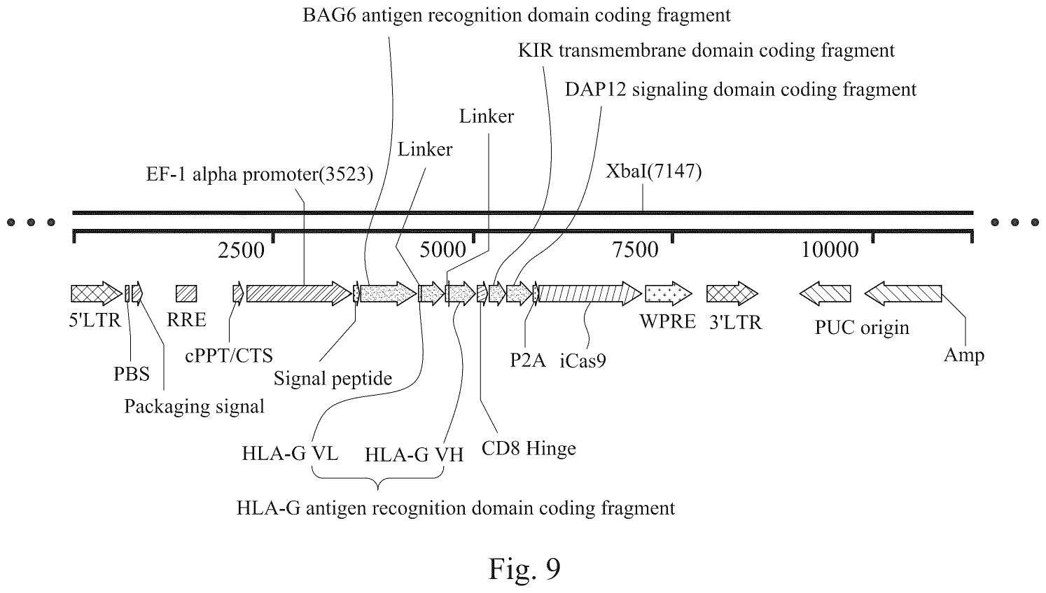

Reference is made to FIG. 9, which is a schematic view showing a construction of a BAG6 specific chimeric antigen receptor expression plasmid according to 2nd embodiment of another aspect of the present disclosure. In detail, the insert of the BAG6 specific chimeric antigen receptor expression plasmid of 2nd embodiment includes in order from a 5′ end to a 3′ end, the promoter including the nucleic acid sequence of SEQ ID NO: 25, the BAG6 antigen recognition domain coding fragment including the nucleic acid sequence of SEQ ID NO: 13, the HLA-G antigen recognition domain coding fragment including the nucleic acid sequence of SEQ ID NO: 14, the KIR transmembrane domain coding fragment including the nucleic acid sequence of SEQ ID NO: 18, the DAP12 signaling domain coding fragment including the nucleic acid sequence of SEQ ID NO: 21, and the suicide gene including the nucleic acid sequence of SEQ ID NO: 24 (iCas9). The HLA-G antigen recognition domain coding fragment includes a HLA-G light chain immunoglobulin coding fragment (represented as HLA-G VL in FIG. 9) and a HLA-G heavy chain immunoglobulin coding fragment (represented as HLA-G VH in FIG. 9). Then, the insert is constructed on Creative Biolabs vector to obtain the BAG6 specific chimeric antigen receptor expression plasmid of 2nd embodiment. The Creative Biolabs vector is a lentivirus vector system, so that the constructed BAG6 specific chimeric antigen receptor expression plasmid of 2nd embodiment can be transfected into expression cells to produce lentiviruses, and the BAG6 specific chimeric antigen receptor expression plasmid of 2nd embodiment can be subsequently transduced into the immune cells using lentiviruses.

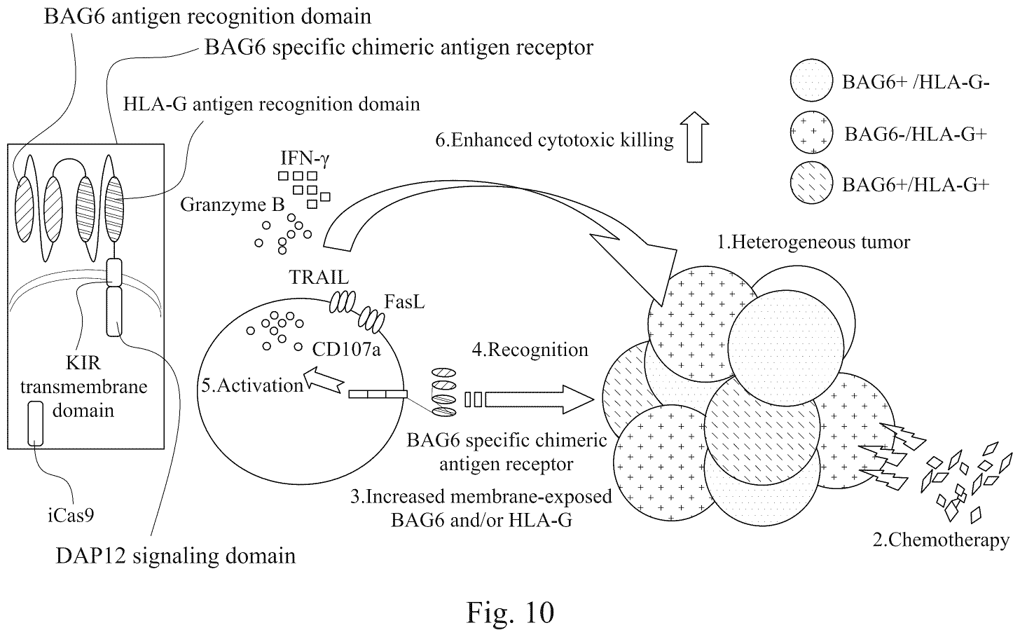

Reference is made to FIG. 10, which is a schematic view showing the theoretical structure and mechanism of the BAG6 specific chimeric antigen receptor according to 2nd embodiment of one aspect of the present disclosure. A BAG6 specific chimeric antigen receptor expressing cell of 2nd embodiment is a genetically engineered NK cell or T cell that expresses the BAG6 specific chimeric antigen receptor of 2nd embodiment, wherein the BAG6 specific chimeric antigen receptor of 2nd embodiment is a tumor-targeting receptor complex consisted of the BAG6 antigen recognition domain, the HLA-G antigen recognition domain, the KIR transmembrane domain and the cytoplasmic domain. The cytoplasmic domain can be the DAP12 signaling domain. Preferably, the BAG6 specific chimeric antigen receptor of 2nd embodiment can further include the suicide protein, and the suicide protein can be iCas9. The BAG6-specific chimeric antigen receptor of 2nd embodiment can specifically recognize BAG6 and/or HLA-G on the tumor cell membrane, thereby killing the tumor cells. Preferably, when the tumor cells are treated with the chemotherapy drug, BAG6 and HLA-G on the cell membrane of the tumor cells can be positively regulated. The BAG6-specific chimeric antigen receptor of 2nd embodiment binds to BAG6 and/or HLA-G specifically recognized on the surface of the tumor cells and triggers signal transduction, resulting in a signal cascade leading to the activation and the proliferation of the BAG6 specific chimeric antigen receptor expressing cell of the present disclosure, thereby triggering exocytosis of lytic granules and killing the target tumor cells.

### 3.1. Example 3

The BAG6 specific chimeric antigen receptor of 2nd embodiment was transduced into the primary T lymphocyte to obtain a BAG6 specific chimeric antigen receptor expressing cell of Example 3 of the present disclosure (hereinafter referred to as Example 3). The effects of Example 3 and the pharmaceutical composition for treating cancer including Example 3 of the present disclosure on inducing the death of the breast cancer cells, the glioblastoma multiforme cells, the pancreatic cancer cells, and the ovarian cancer cells were further demonstrated in following experiments.

First, the human breast cancer cell line MDA-MB-231, the human malignant brain tumor cell line DBTRG, the human pancreatic cancer cell line AsPC-1 and the human ovarian cancer cell line SKOV3 were seeded in a 12-well plate at a density of 1×10⁵ cells/well. The cells were subsequently incubated for 48 hours. Each type of the tumor cells was divided into six groups. In a control, the tumor cells were untreated. In a group 1, the tumor cells were treated with the chemotherapy drug. In a group 2, the tumor cells were treated with the parental primary T lymphocyte. In a group 3, the tumor cells were treated with the parental primary T lymphocyte and the chemotherapy drug. In a group 4, the tumor cells were treated with Example 3. In a group 5, the tumor cells were treated with Example 3 and the chemotherapy drug. The chemotherapy drug used for treating the human breast cancer cell line MDA-MB-231 was doxorubicin (200 nM), the chemotherapy drug used for treating the human malignant brain tumor cell line DBTRG was temozolomide (80 μg/mL), the chemotherapy drug used for treating the human pancreatic cancer cell line AsPC-1 was gemcitabine (20 μM), and the chemotherapy drug used for treating the human ovarian cancer cell line SKOV3 was carboplatin (20 μM). In the group 4 and the group 5, the number of Example 3 treated was 1×10⁵ cells. In the group 2 and the group 3, the number of the parental primary T lymphocyte treated was 1×10⁵ cells. The treated cells of each group were stained with Annexin V-FITC and PI, and the apoptosis and the death of the tumor cells were detected by the flow cytometry. The sum of the percentage of cells stained with Annexin V-FITC and/or PI were calculated to obtain the cytotoxicity. The results of the cytotoxicity were counted after three independent trials in each group.

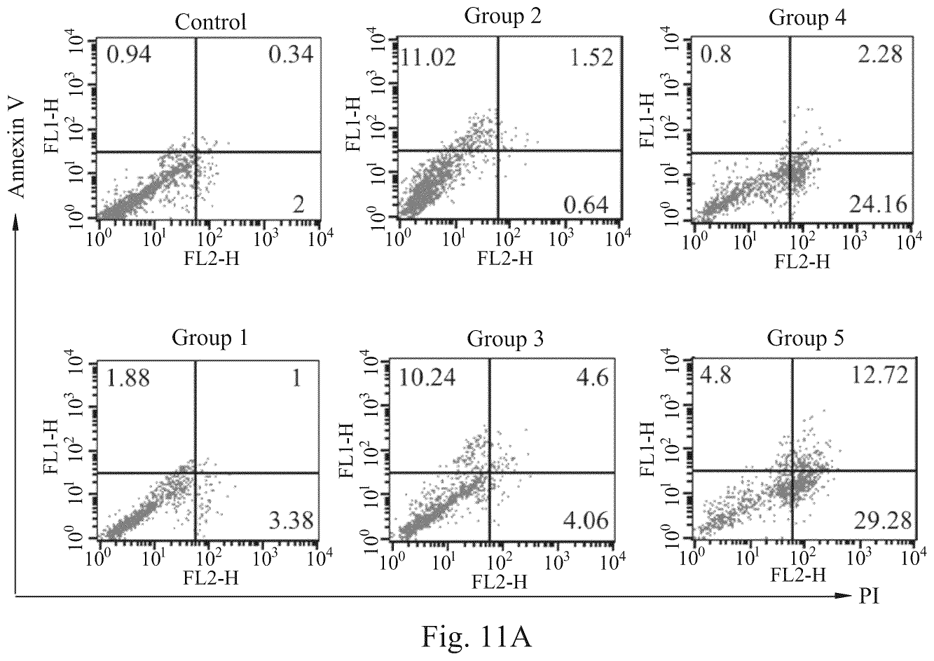

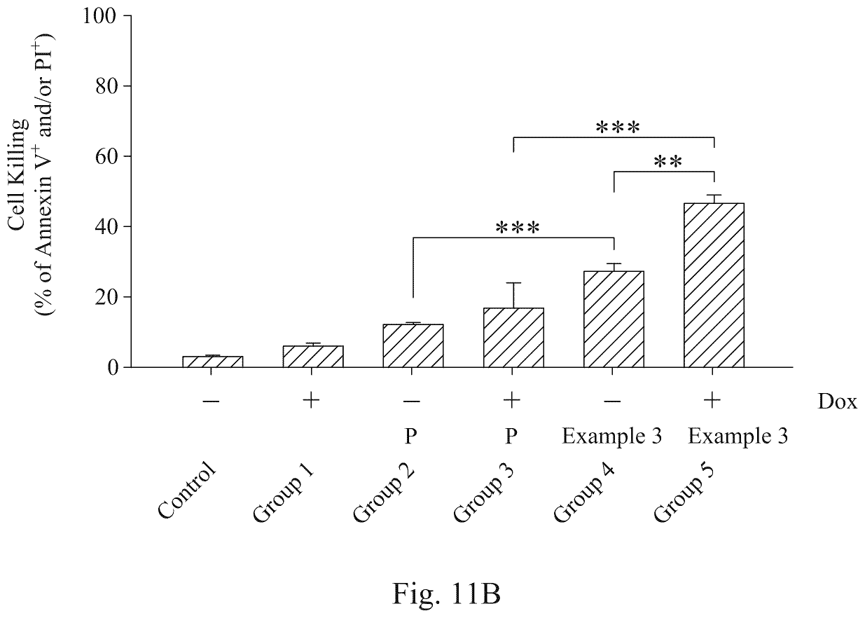

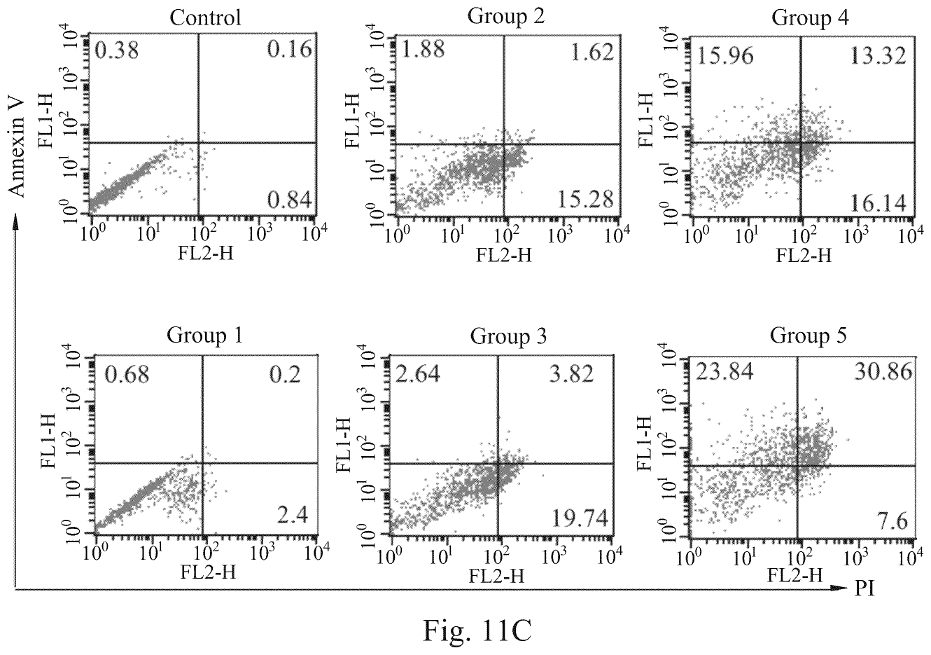

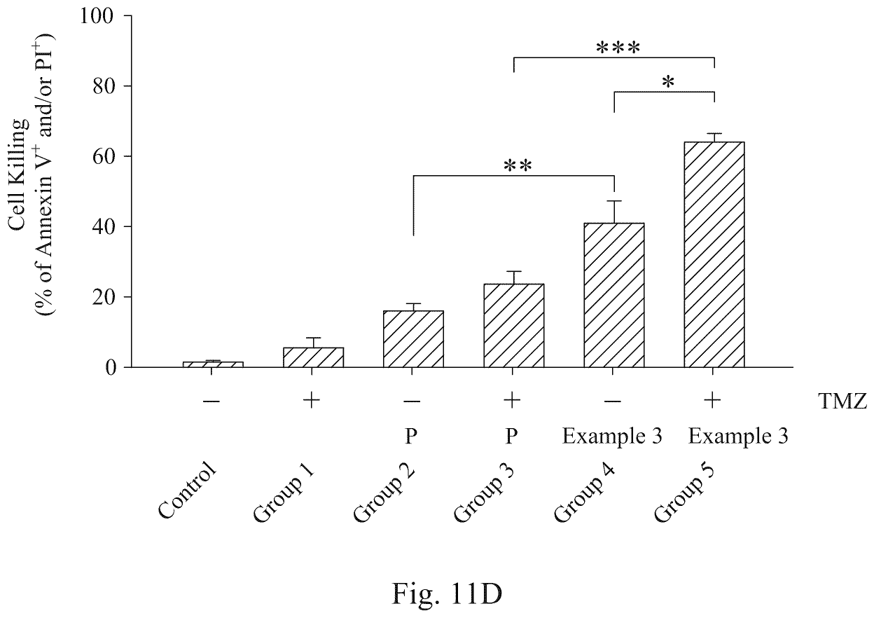

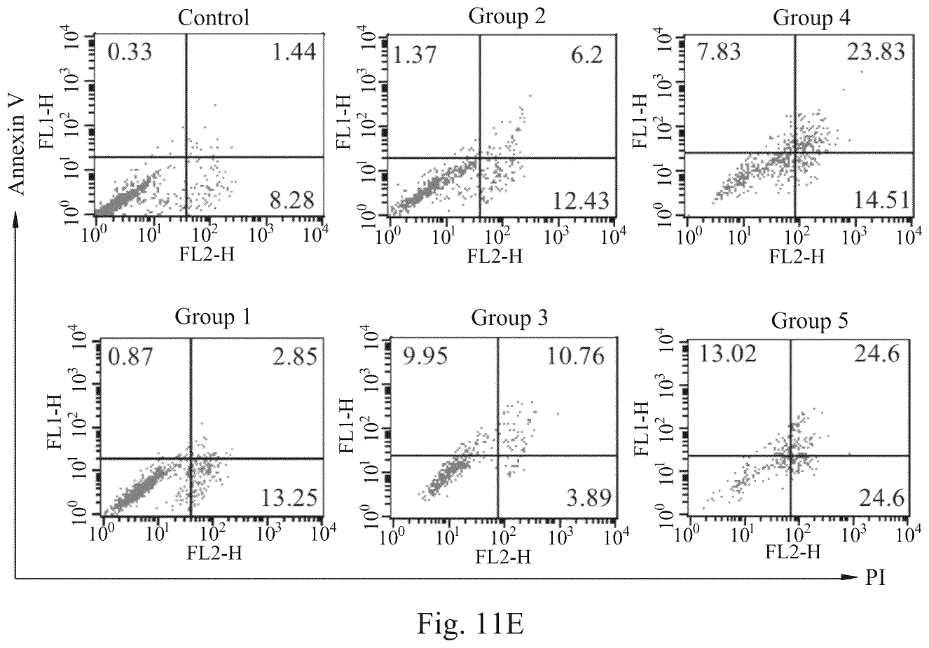

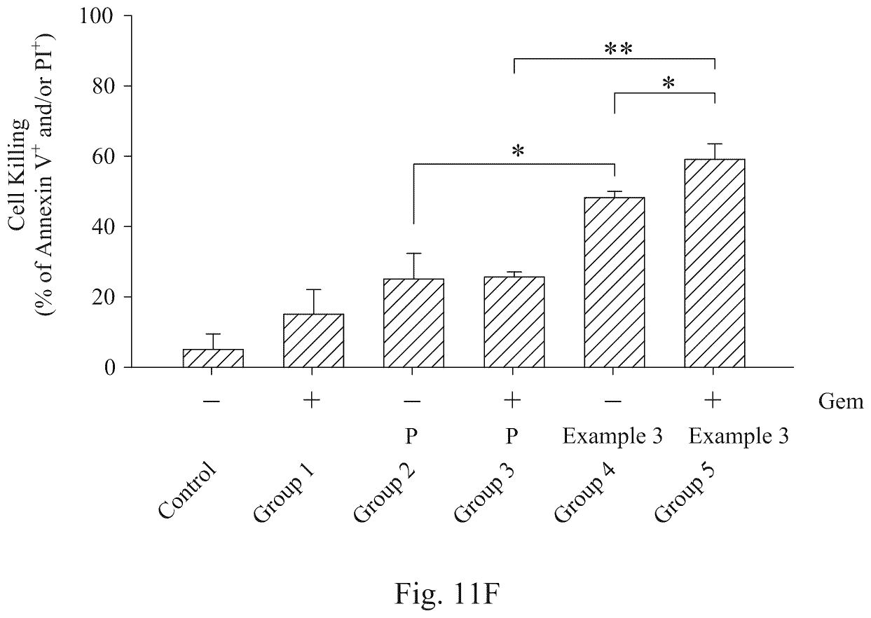

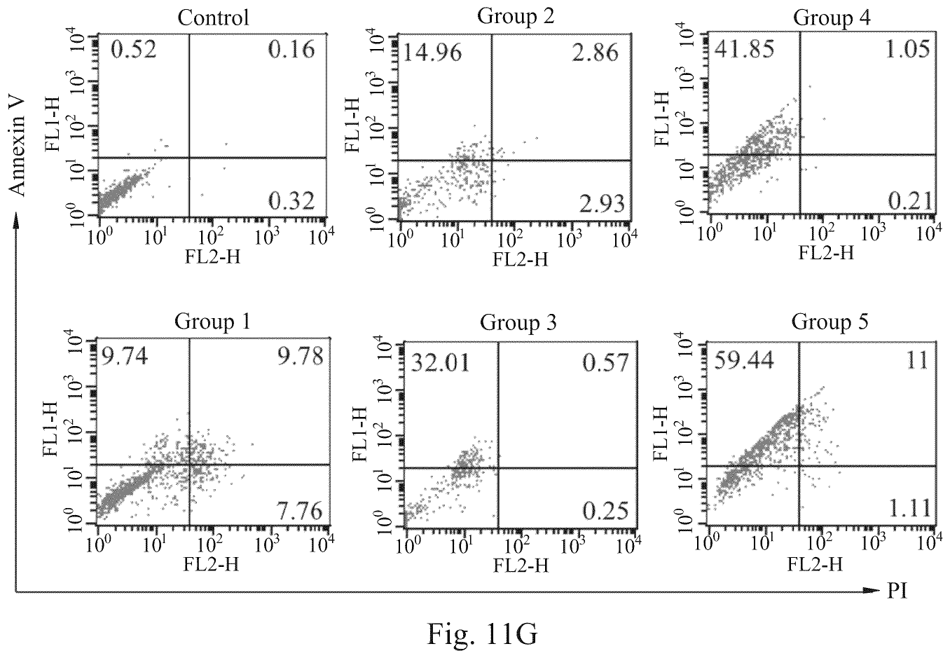

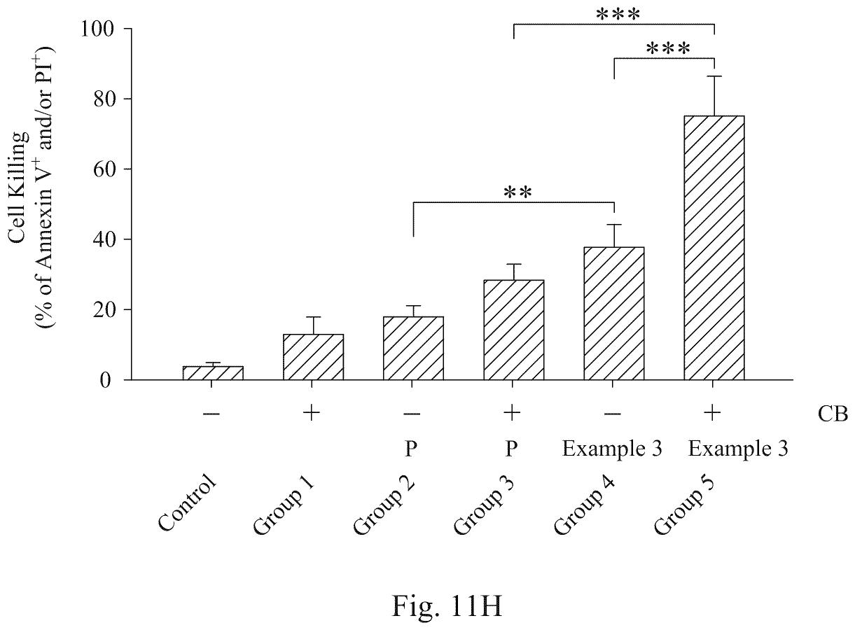

Reference is made to FIG. 11A, FIG. 11B, FIG. 11C, FIG. 11D, FIG. 11E, FIG. 11F, FIG. 11G, FIG. 11H and Table 3, which show analytical results of tumor cell death induced by the BAG6 specific chimeric antigen receptor expressing cells according to Example 3 of the present disclosure, wherein “P” represents parental primary T cells in FIG. 11B, FIG. 11D, FIG. 11F and FIG. 11H. FIG. 11A is a graph showing the analytical results of the human breast cancer cell line MDA-MB-231, and FIG. 11B is a statistical chart of FIG. 11A after three independent trials, wherein “Dox” represents doxorubicin. FIG. 11C is a graph showing the analytical results of the human malignant brain tumor cell line DBTRG, and FIG. 11D is a statistical chart of FIG. 11C after three independent trials, wherein “TMZ” represents temozolomide. FIG. 11E is a graph showing the analytical results of the human pancreatic cancer cell line AsPC-1, and FIG. 11F is a statistical chart of FIG. 11E after three independent trials, wherein “Gem” represents gemcitabine. FIG. 11G is a graph showing the analytical results of the human ovarian cancer cell line SKOV3, and FIG. 11H is a statistical chart of FIG. 11G after three independent trials, wherein “CB” represents carboplatin. Table 3 shows the death rate of each group.

TABLE 3
Analytical results of Example 3

| | Control | Group 1 | Group 2 | Group 3 | Group 4 | Group 5 |
|---|---|---|---|---|---|---|
| MDA- | 3.0767 ± | 6.0133 ± | 12.1467 ± | 16.7733 ± | 27.2733 ± | 46.5633 ± |
| MB-231 | 0.3092 | 0.8764 | 0.5836 | 7.2169 | 2.1902 | 2.4366 |
| DBTRG | 1.5200 ± | 5.5450 ± | 15.9600 ± | 23.6950 ± | 40.9100 ± | 64.0200 ± |
| | 0.4205 | 2.8199 | 2.1332 | 3.5835 | 6.3781 | 2.4324 |
| AsPC-1 | 5.0533 ± | 15.0933 ± | 25.1500 ± | 25.6650 ± | 48.2300 ± | 59.0700 ± |
| | 4.4052 | 7.0031 | 7.2832 | 1.5061 | 1.7912 | 4.4548 |
| SKOV3 | 3.8000 ± | 12.9489 ± | 17.9583 ± | 28.3817 ± | 37.7233 ± | 75.1533 ± |
| | 1.1257 | 4.9276 | 3.1414 | 4.5713 | 6.4747 | 11.2370 |

The results of Table 3, FIG. 11A and FIG. 11B show that in the human breast cancer cell line MDA-MB-231, the death rate of the human breast cancer cell line MDA-MB-231 in the group 4 treated with Example 3 was approximately 27.3%, and there was a statistically significant difference (p<0.001) compared to the group 2. In addition, the death rate of the human breast cancer cell line MDA-MB-231 in the group 5 treated with Example 3 and doxorubicin could reach 46.6%, and there was a statistically significant difference (p<0.01) compared to the group 4 and a statistically significant difference (p<0.001) compared to the group 3, respectively.

The results of Table 3, FIG. 11C and FIG. 11D show that in the human malignant brain tumor cell line DBTRG, the death rate of the human malignant brain tumor cell line DBTRG in the group 4 treated with Example 3 was about 40.9%, and there was a statistically significant difference (p<0.01) compared to the group 2. In addition, the death rate of the human malignant brain tumor cell line DBTRG in the group 5 treated with Example 3 and temozolomide was over 64%, and there was a statistically significant difference (p<0.05) compared to the group 4 and a statistically significant difference (p<0.001) compared to the group 3, respectively.

The results of Table 3, FIG. 11E and FIG. 11F show that in the human pancreatic cancer cell line AsPC-1, the death rate of the human pancreatic cancer cell line AsPC-1 in the group 4 treated with Example 3 was about 48.2%, and there was a statistically significant difference (p<0.05) compared to the group 2. In addition, the death rate of the human pancreatic cancer cell line AsPC-1 in the group 5 treated with Example 3 and gemcitabine was 59.1%, and there was a statistically significant difference (p<0.05) compared to the group 4 and a statistically significant difference (p<0.01) compared to the group 3, respectively.

The results of Table 3, FIG. 11G and FIG. 11H show that in the human ovarian cancer cell line SKOV3, the death rate of the human ovarian cancer cell line SKOV3 in the group 4 treated with Example 3 was about 37.7%, and there was a statistically significant difference (p<0.01) compared to the group 2. In addition, the death rate of the human ovarian cancer cell line SKOV3 in the group 5 treated with Example 3 and carboplatin could reach 75.2%, and there was a statistically significant difference (p<0.001) compared to the group 4 and a statistically significant difference (p<0.001) compared to the group 3, respectively.

### 3.2. Example 4

The BAG6 specific chimeric antigen receptor of 2nd embodiment was transduced into the primary NK cell to obtain a BAG6 specific chimeric antigen receptor expressing cell of Example 4 of the present disclosure (hereinafter referred to as Example 4). The effects of Example 4 and the pharmaceutical composition for treating cancer including Example 4 of the present disclosure on inducing the death of the breast cancer cells, the glioblastoma multiforme cells, the pancreatic cancer cells, and the ovarian cancer cells were further demonstrated in following experiments.

First, the human breast cancer cell line MDA-MB-231, the human malignant brain tumor cell line DBTRG, the human pancreatic cancer cell line AsPC-1 and the human ovarian cancer cell line SKOV3 were seeded in a 12-well plate at a density of 1×10⁵ cells/well. The cells were subsequently incubated for 48 hours. Each type of the tumor cells was divided into six groups. In a control, the tumor cells were untreated. In a group 1, the tumor cells were treated with the chemotherapy drug. In a group 2, the tumor cells were treated with the parental primary NK cell. In a group 3, the tumor cells were treated with the parental primary NK cell and the chemotherapy drug. In a group 4, the tumor cells were treated with Example 4. In a group 5, the tumor cells were treated with Example 4 and the chemotherapy drug. The chemotherapy drug used for treating the human breast cancer cell line MDA-MB-231 was doxorubicin (200 nM), the chemotherapy drug used for treating the human malignant brain tumor cell line DBTRG was temozolomide (80 μg/mL), the chemotherapy drug used for treating the human pancreatic cancer cell line AsPC-1 was gemcitabine (20 μM), and the chemotherapy drug used for treating the human ovarian cancer cell line SKOV3 was carboplatin (20 μM). In the group 4 and the group 5, the number of Example 4 treated was 1×10⁵ cells. In the group 2 and the group 3, the number of the parental primary NK cell treated was 1×10⁵ cells. The treated cells of each group were stained with Annexin V-FITC and PI, and the apoptosis and the death of the tumor cells were detected by the flow cytometry. The sum of the percentage of cells stained with Annexin V-FITC and/or PI were calculated to obtain the cytotoxicity. The results of the cytotoxicity were counted after three independent trials in each group.

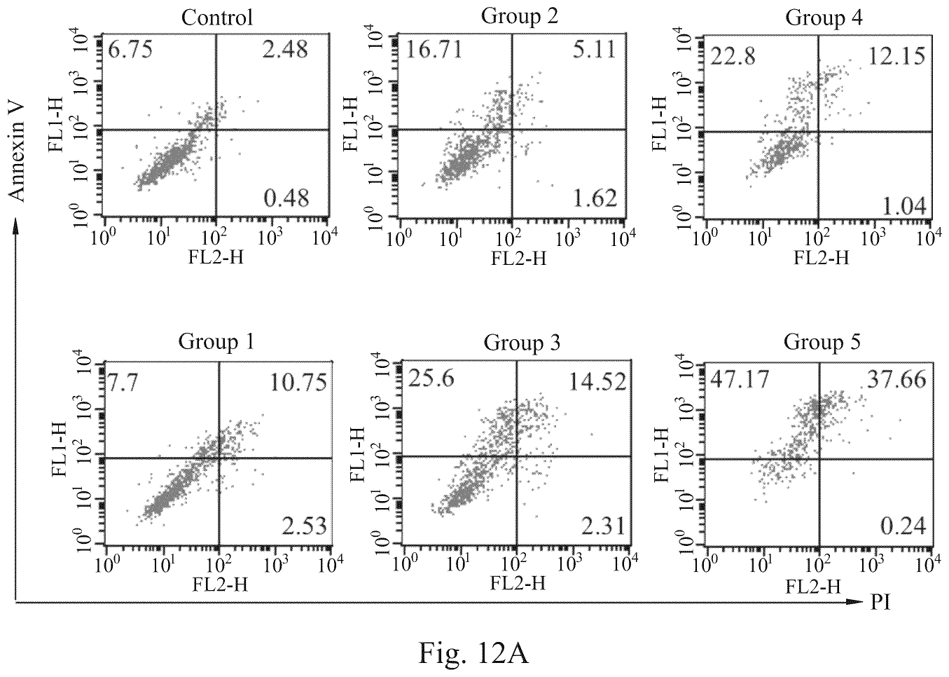

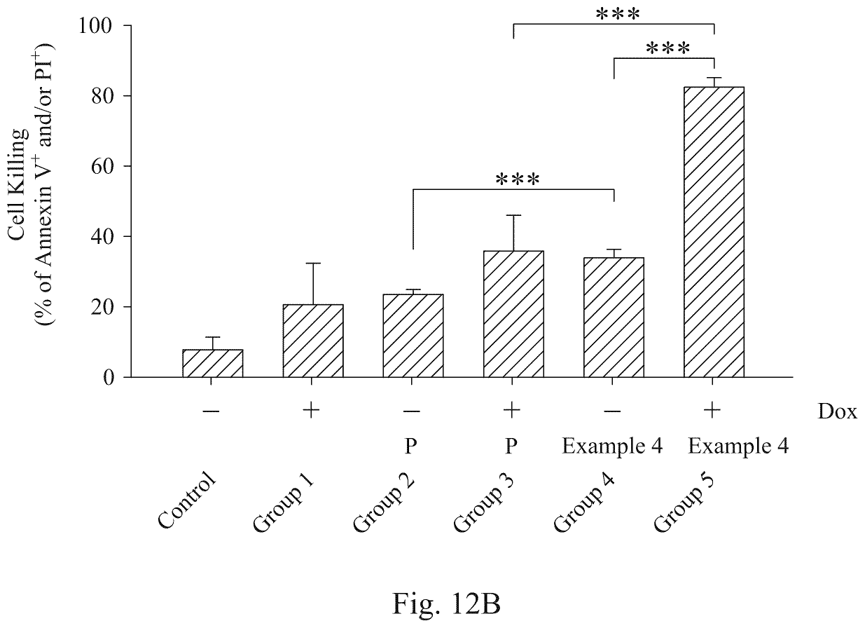

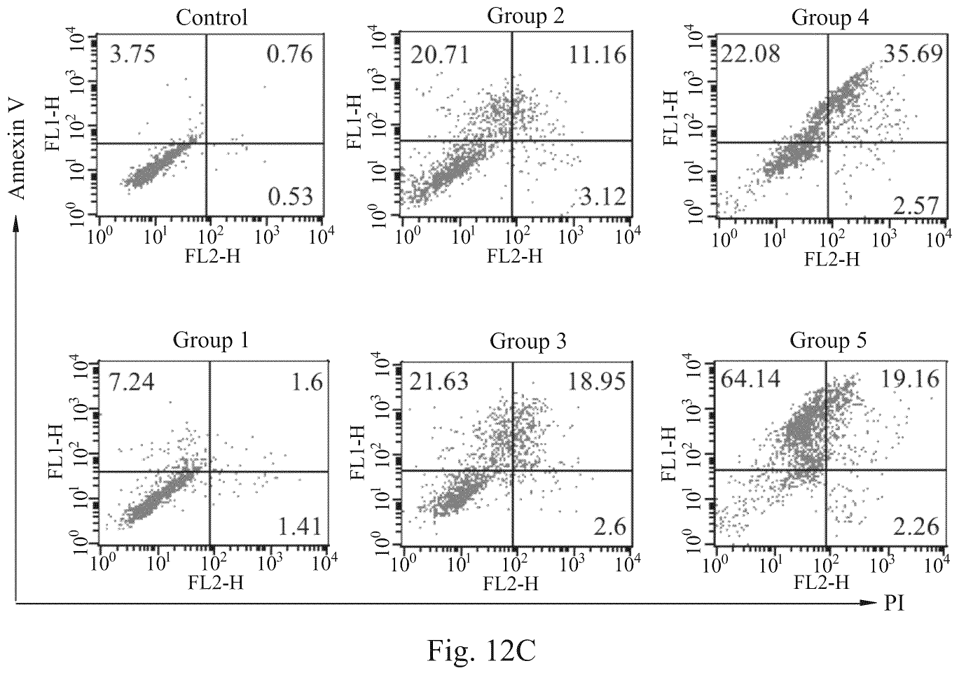

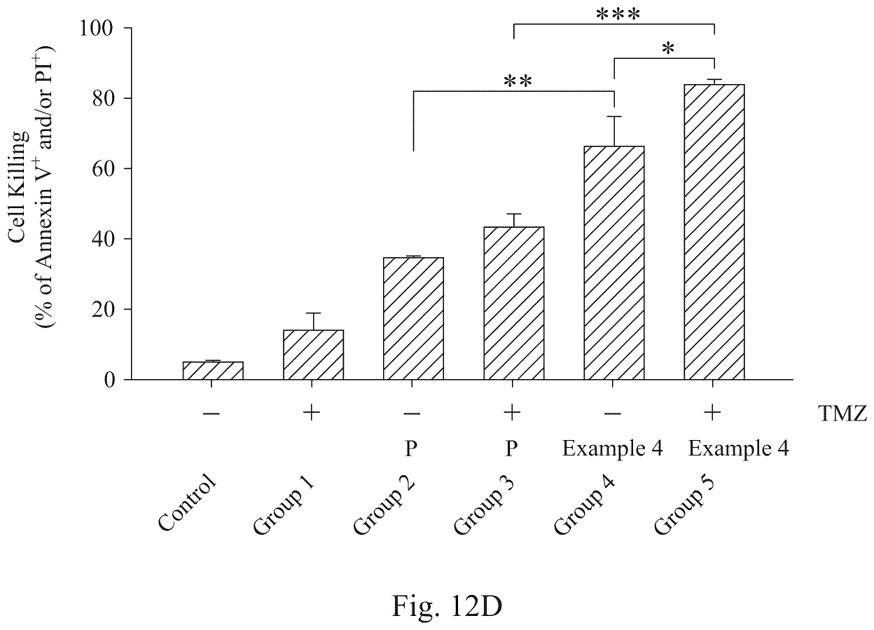

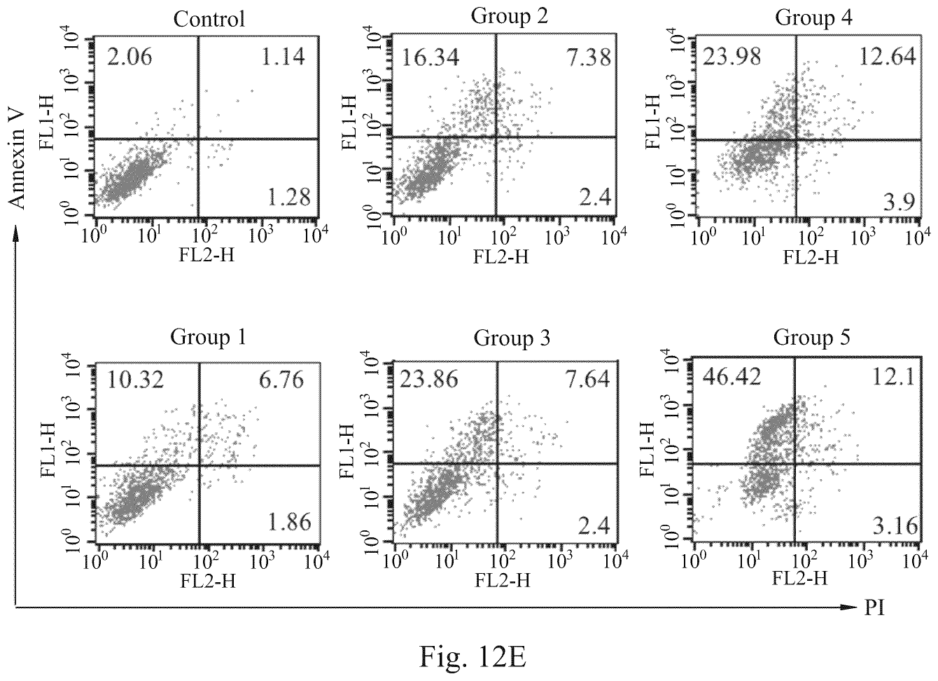

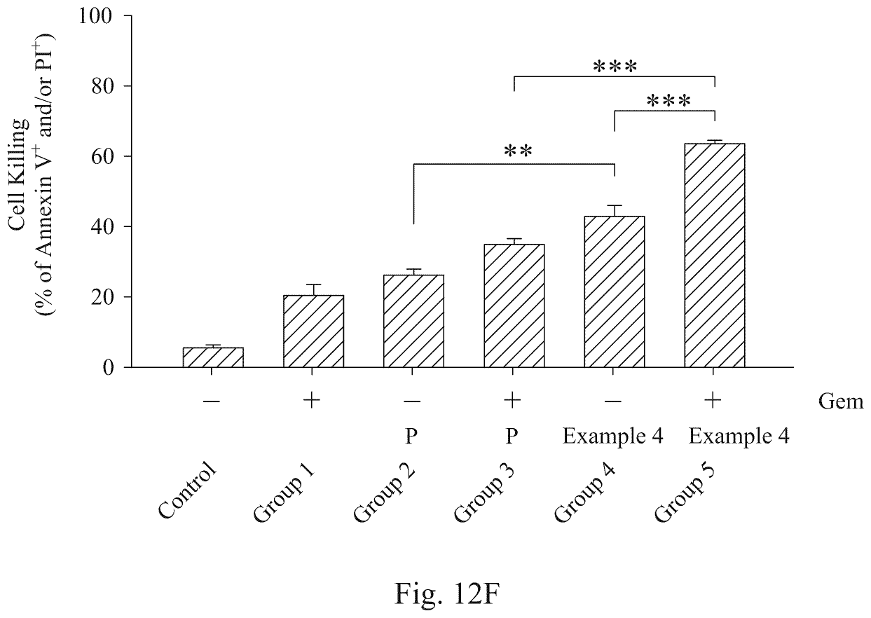

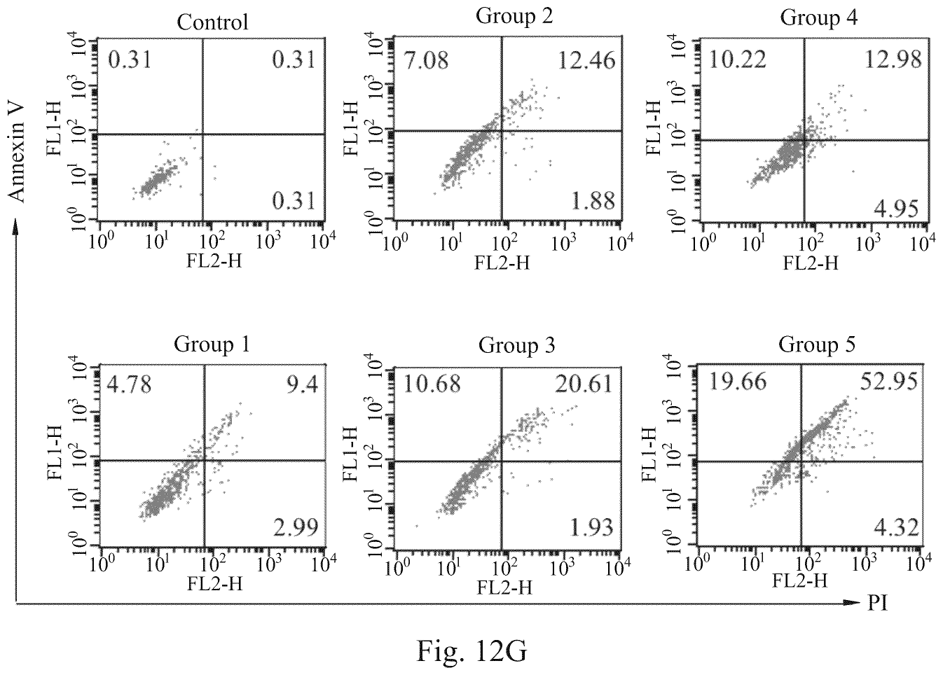

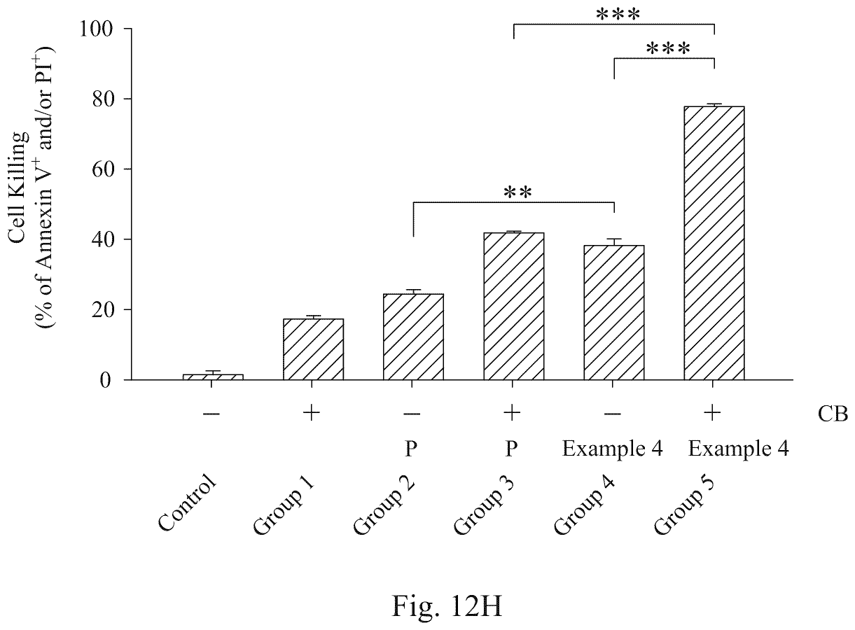

Reference is made to FIG. 12A, FIG. 12B, FIG. 12C, FIG. 12D, FIG. 12E, FIG. 12F, FIG. 12G, FIG. 12H and Table 4, which show analytical results of tumor cell death induced by the BAG6 specific chimeric antigen receptor expressing cells according to Example 4 of the present disclosure, wherein “P” represents parental primary NK cell in FIG. 12B, FIG. 12D, FIG. 12F and FIG. 12H. FIG. 12A is a graph showing the analytical results of the human breast cancer cell line MDA-MB-231, and FIG. 12B is a statistical chart of FIG. 12A after three independent trials, wherein “Dox” represents doxorubicin. FIG. 12C is a graph showing the analytical results of the human malignant brain tumor cell line DBTRG, and FIG. 12D is a statistical chart of FIG. 12C after three independent trials, wherein “TMZ” represents temozolomide. FIG. 12E is a graph showing the analytical results of the human pancreatic cancer cell line AsPC-1, and FIG. 12F is a statistical chart of FIG. 12E after three independent trials, wherein “Gem” represents gemcitabine. FIG. 12G is a graph showing the analytical results of the human ovarian cancer cell line SKOV3, and FIG. 12H is a statistical chart of FIG. 12G after three independent trials, wherein “CB” represents carboplatin. Table 4 shows the death rate of each group.

TABLE 4
Analytical results of Example 4

| | Control | Group 1 | Group 2 | Group 3 | Group 4 | Group 5 |
|---|---|---|---|---|---|---|
| MDA- | 7.7600 ± | 20.6575 ± | 23.4650 ± | 35.8425 ± | 33.8767 ± | 82.4267 ± |
| MB-231 | 3.6169 | 11.7058 | 1.4557 | 10.2273 | 2.4310 | 2.7674 |
| DBTRG | 5.0067 ± | 11.7933 ± | 37.2933 ± | 41.4500 ± | 66.3250 ± | 83.800 ± |
| | 0.5397 | 2.7865 | 4.7175 | 2.4466 | 8.464 | 1.5427 |
| AsPC-1 | 5.4600 ± | 20.3533 ± | 26.1733 ± | 34.9067 ± | 42.8600 ± | 63.5500 ± |
| | 0.8793 | 3.1293 | 1.7206 | 1.6070 | 3.1031 | 0.9758 |
| SKOV3 | 1.5433 ± | 17.3000 ± | 24.3750 ± | 41.8450 ± | 38.2550 ± | 77.7400 ± |
| | 1.0193 | 1.0133 | 1.3223 | 0.5162 | 1.9021 | 0.8050 |

The results of Table 4, FIG. 12A and FIG. 12B show that in the human breast cancer cell line MDA-MB-231, the death rate of the human breast cancer cell line MDA-MB-231 in the group 4 treated with Example 4 was 33.9%, and there was a statistically significant difference (p<0.001) compared to the group 2. In addition, the death rate of the human breast cancer cell line MDA-MB-231 in the group 5 treated with Example 4 and doxorubicin was over 80%, and there was a statistically significant difference (p<0.001) compared to the group 4 and a statistically significant difference (p<0.001) compared to the group 3, respectively.

The results of Table 4, FIG. 12C and FIG. 12D show that in the human malignant brain tumor cell line DBTRG, the death rate of the human malignant brain tumor cell line DBTRG in the group 4 treated with Example 4 was about 66.3%, and there was a statistically significant difference (p<0.01) compared to the group 2. In addition, the death rate of the human malignant brain tumor cell line DBTRG in the group 5 treated with Example 4 and temozolomide could reach 83.8%, and there was a statistically significant difference (p<0.05) compared to the group 4 and a statistically significant difference (p<0.001) compared to the group 3, respectively.

The results of Table 4, FIG. 12E and FIG. 12F show that in the human pancreatic cancer cell line AsPC-1, the death rate of the human pancreatic cancer cell line AsPC-1 in the group 4 treated with Example 4 was about 42.9%, and there was a statistically significant difference (p<0.01) compared to the group 2. In addition, the death rate of the human pancreatic cancer cell line AsPC-1 in the group 5 treated with Example 4 and gemcitabine could reach 63.6%, and there was a statistically significant difference (p<0.001) compared to the group 4 and a statistically significant difference (p<0.001) compared to the group 3, respectively.

The results of Table 4, FIG. 12G and FIG. 12H show that in the human ovarian cancer cell line SKOV3, the death rate of the human ovarian cancer cell line SKOV3 in the group 4 treated with Example 4 was about 38.3%, and there was a statistically significant difference (p<0.01) compared to the group 2. In addition, the death rate of the human ovarian cancer cell line SKOV3 in the group 5 treated with Example 4 and carboplatin could reach 77.7%, and there was a statistically significant difference (p<0.001) compared to the group 4 and a statistically significant difference (p<0.001) compared to the group 3, respectively.

The results of Table 3, Table 4 and FIG. 11A to FIG. 12H show that the BAG6 specific chimeric antigen receptor expressing cell of 2nd embodiment can be used to treat with the breast cancer cell, the glioblastoma multiforme cell, the pancreatic cancer cell or the ovarian cancer cell for excellent cell killing. Therefore, the BAG6 specific chimeric antigen receptor expressing cell of the present disclosure can be used for inhibiting the proliferation of the tumor cells in the subject in need for the treatment of the tumor. In addition, the results of FIG. 2A, FIG. 2B and FIG. 3 show that treatment with the chemotherapy drug increased the expression of BAG6 and the expression of HLA-G on the cell membrane of the tumor cells, and the BAG6 specific chimeric antigen receptor expressed by the BAG6 specific chimeric antigen receptor expressing cell of 2nd embodiment can specifically bind to BAG6 and HLA-G. Therefore, the treatment of treating the chemotherapy drug first and then treating the BAG6 specific chimeric antigen receptor expressing cell of 2nd embodiment, or the simultaneous treatment of the chemotherapy drug and the BAG6 specific chimeric antigen receptor expressing cell of 2nd embodiment can achieve a better effect on killing the tumor cells. The results indicate that the pharmaceutical composition for treating cancer of the present disclosure can effectively inhibit the growth of the tumor cells and treat cancer. Preferably, the pharmaceutical composition for treating cancer can include the chemotherapy drug.

### IV. 3rd Embodiment

A BAG6 specific chimeric antigen receptor of 3rd embodiment includes, in order from an N-terminus to a C-terminus, the BAG6 antigen recognition domain including the amino acid sequence of SEQ ID NO: 1, the B7-H6 antigen recognition domain including the amino acid sequence of SEQ ID NO: 3, the CD8 transmembrane domain including the amino acid sequence of SEQ ID NO: 5, the 4-1BB signaling domain including the amino acid sequence of SEQ ID NO: 11, and the CD3ζ signaling domain including the amino acid sequence of SEQ ID NO: 10. In addition, the BAG6 specific chimeric antigen receptor of 3rd embodiment further includes the suicide protein including the amino acid sequence of SEQ ID NO: 12, which is linked to the C-terminus of the CD3ζ signaling domain. A nucleic acid of 3rd embodiment includes, in order from a 5′ end to a 3′ end, the BAG6 antigen recognition domain coding fragment including the nucleic acid sequence of SEQ ID NO: 13, the B7-H6 antigen recognition domain coding fragment including the nucleic acid sequence of SEQ ID NO: 15, the CD8 transmembrane domain coding fragment including the nucleic acid sequence of SEQ ID NO: 17, the 4-1BB signaling domain coding fragment including the nucleic acid sequence of SEQ ID NO: 23, and the CD3ζ signaling domain coding fragment including the nucleic acid sequence of SEQ ID NO: 22. In addition, the nucleic acid of 3rd embodiment further includes the suicide gene including the nucleic acid sequence of SEQ ID NO: 24, which is linked to the 3′ end of the CD3ζ signaling domain coding fragment.

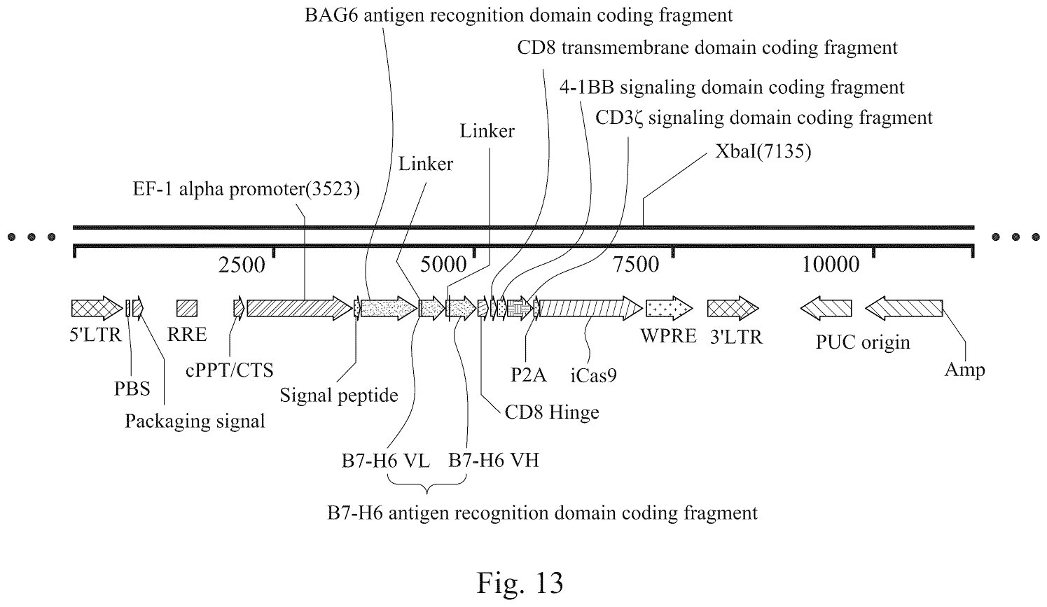

Reference is made to FIG. 13, which is a schematic view showing a construction of a BAG6 specific chimeric antigen receptor expression plasmid according to 3rd embodiment of another aspect of the present disclosure. In detail, the insert of the BAG6 specific chimeric antigen receptor expression plasmid of 3rd embodiment includes in order from a 5′ end to a 3′ end, the promoter including the nucleic acid sequence of SEQ ID NO: 25, the BAG6 antigen recognition domain coding fragment including the nucleic acid sequence of SEQ ID NO: 13, the B7-H6 antigen recognition domain coding fragment including the nucleic acid sequence of SEQ ID NO: 15, the CD8 transmembrane domain coding fragment including the nucleic acid sequence of SEQ ID NO: 17, the cytoplasmic domain coding fragment, and the suicide gene including the nucleic acid sequence of SEQ ID NO: 24 (iCas9). The B7-H6 antigen recognition domain coding fragment includes a B7-H6 light chain immunoglobulin coding fragment (represented as B7-H6 VL in FIG. 13) and a B7-H6 heavy chain immunoglobulin coding fragment (represented as B7-H6 VH in FIG. 13). The cytoplasmic domain coding fragment includes the 4-1BB signaling domain coding fragment including the nucleic acid sequence of SEQ ID NO: 23, and the CD3ζ signaling domain coding fragment including the nucleic acid sequence of SEQ ID NO: 22. Then, the insert is constructed on Creative Biolabs vector to obtain the BAG6 specific chimeric antigen receptor expression plasmid of 3rd embodiment. The Creative Biolabs vector is a lentivirus vector system, so that the constructed BAG6 specific chimeric antigen receptor expression plasmid of 3rd embodiment can be transfected into expression cells to produce lentiviruses, and the BAG6 specific chimeric antigen receptor expression plasmid of 3rd embodiment can be subsequently transduced into the immune cells using lentiviruses.

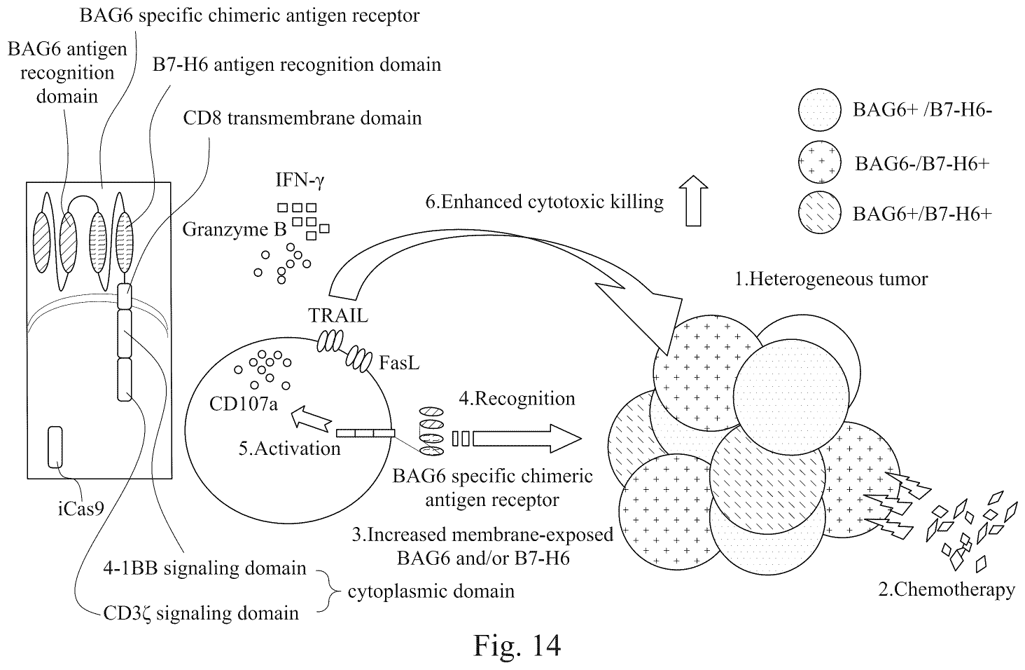

Reference is made to FIG. 14, which is a schematic view showing the theoretical structure and mechanism of the BAG6 specific chimeric antigen receptor according to 3rd embodiment of one aspect of the present disclosure. A BAG6 specific chimeric antigen receptor expressing cell of 3rd embodiment is a genetically engineered NK cell or T cell that expresses the BAG6 specific chimeric antigen receptor of 3rd embodiment, wherein the BAG6 specific chimeric antigen receptor of 3rd embodiment is a tumor-targeting receptor complex consisted of the BAG6 antigen recognition domain, the B7-H6 antigen recognition domain, the CD8 transmembrane domain and the cytoplasmic domain. The cytoplasmic domain includes the 4-1BB signaling domain and the CD3ζ signaling domain. Preferably, the BAG6 specific chimeric antigen receptor of 3rd embodiment can further include the suicide protein, and the suicide protein can be iCas9. The BAG6-specific chimeric antigen receptor of 3rd embodiment can specifically recognize BAG6 and/or B7-H6 on the tumor cell membrane, thereby killing the tumor cells. Preferably, when the tumor cells are treated with the chemotherapy drug, BAG6 and B7-H6 on the cell membrane of the tumor cells can be positively regulated. The BAG6-specific chimeric antigen receptor of 3rd embodiment binds to BAG6 and/or B7-H6 specifically recognized on the surface of the tumor cells and triggers signal transduction, resulting in a signal cascade leading to the activation and the proliferation of the BAG6 specific chimeric antigen receptor expressing cell of the present disclosure, thereby triggering exocytosis of lytic granules and killing the target tumor cells.

### 4.1. Example 5

The BAG6 specific chimeric antigen receptor of 3rd embodiment was transduced into the primary T lymphocyte to obtain a BAG6 specific chimeric antigen receptor expressing cell of Example 5 of the present disclosure (hereinafter referred to as Example 5). The effects of Example 5 and the pharmaceutical composition for treating cancer including Example 5 of the present disclosure on inducing the death of the breast cancer cells, the glioblastoma multiforme cells, the pancreatic cancer cells, and the ovarian cancer cells were further demonstrated in following experiments.

First, the human breast cancer cell line MDA-MB-231, the human malignant brain tumor cell line DBTRG, the human pancreatic cancer cell line AsPC-1 and the human ovarian cancer cell line SKOV3 were seeded in a 12-well plate at a density of 1×10⁵ cells/well. The cells were subsequently incubated for 48 hours. Each type of the tumor cells was divided into six groups. In a control, the tumor cells were untreated. In a group 1, the tumor cells were treated with the chemotherapy drug. In a group 2, the tumor cells were treated with the parental primary T lymphocyte. In a group 3, the tumor cells were treated with the parental primary T lymphocyte and the chemotherapy drug. In a group 4, the tumor cells were treated with Example 5. In a group 5, the tumor cells were treated with Example 5 and the chemotherapy drug. The chemotherapy drug used for treating the human breast cancer cell line MDA-MB-231 was doxorubicin (200 nM), the chemotherapy drug used for treating the human malignant brain tumor cell line DBTRG was temozolomide (80 μg/mL), the chemotherapy drug used for treating the human pancreatic cancer cell line AsPC-1 was gemcitabine (20 μM), and the chemotherapy drug used for treating the human ovarian cancer cell line SKOV3 was carboplatin (20 μM). In the group 4 and the group 5, the number of Example 5 treated was 1×10⁵ cells. In the group 2 and the group 3, the number of the parental primary T lymphocyte treated was 1×10⁵ cells. The treated cells of each group were stained with Annexin V-FITC and PI, and the apoptosis and the death of the tumor cells were detected by the flow cytometry. The sum of the percentage of cells stained with Annexin V-FITC and/or PI were calculated to obtain the cytotoxicity. The results of the cytotoxicity were counted after three independent trials in each group.

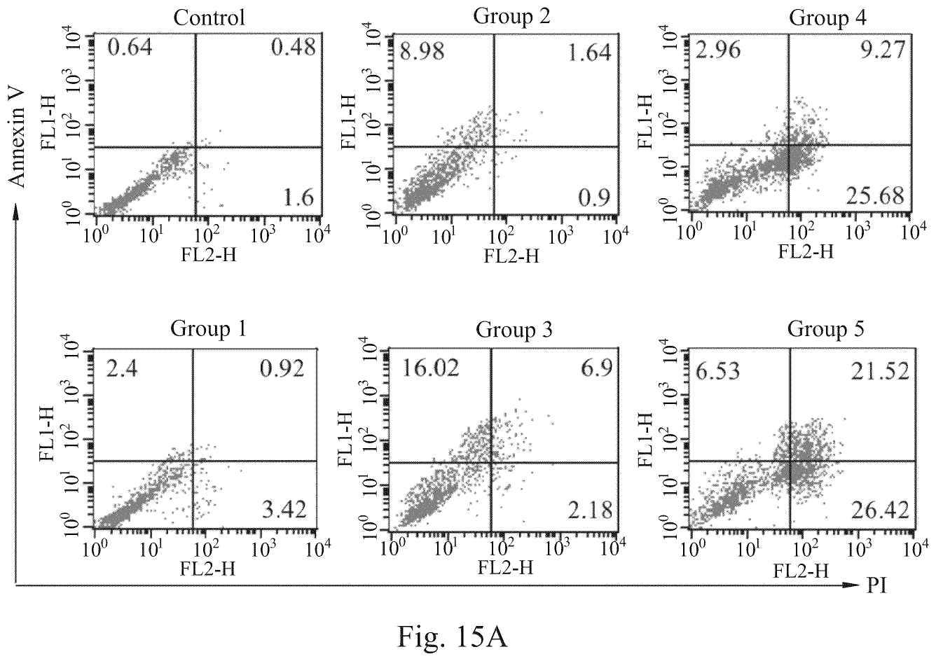

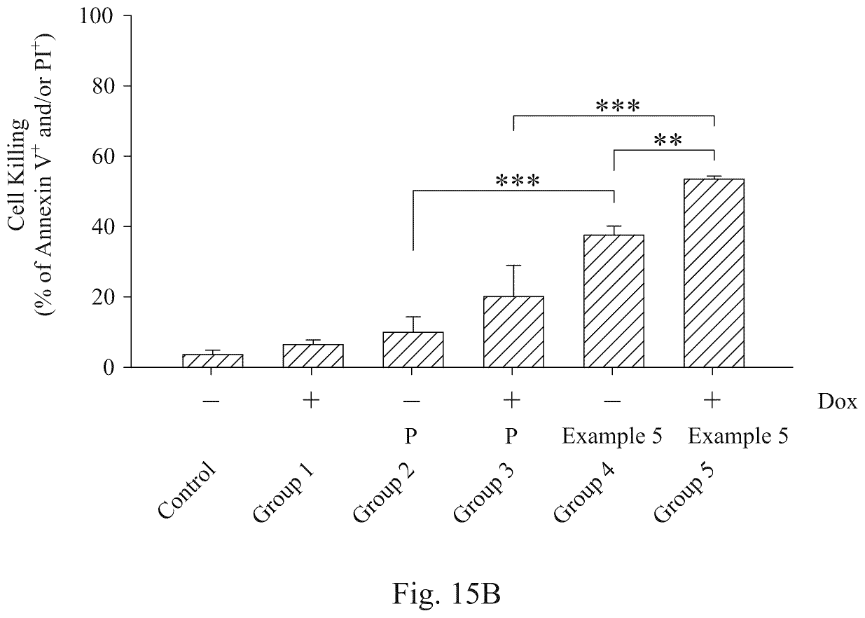

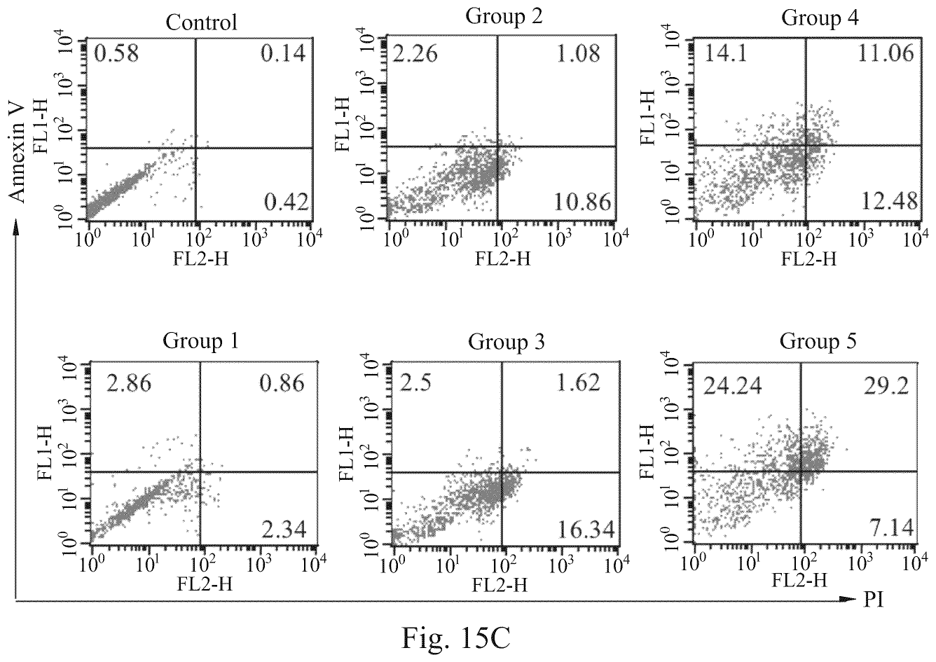

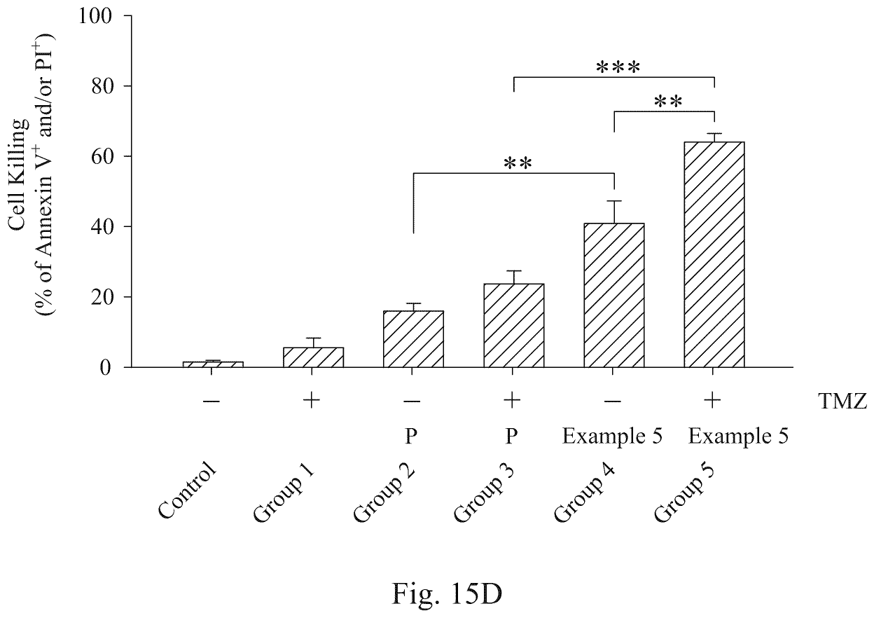

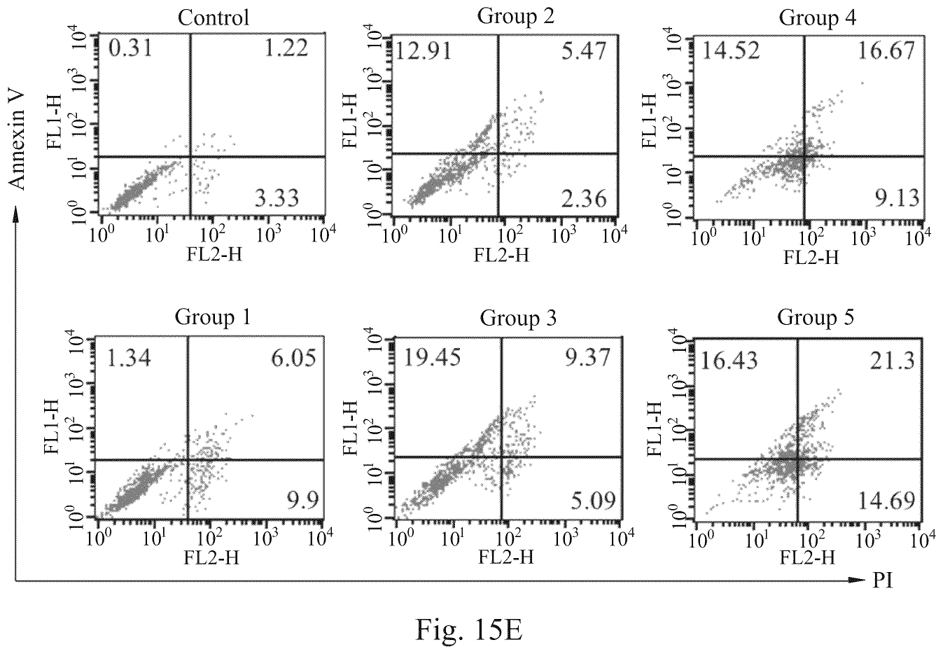

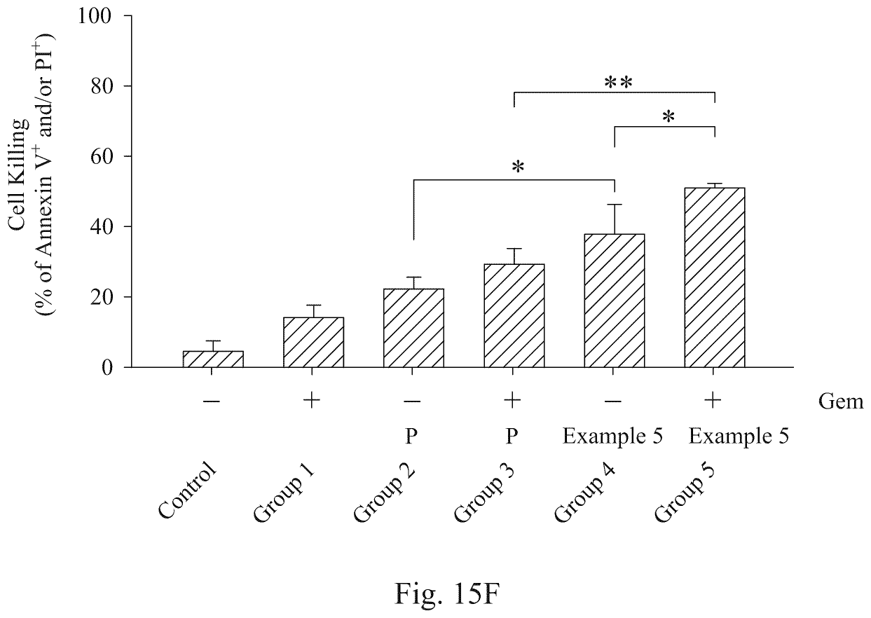

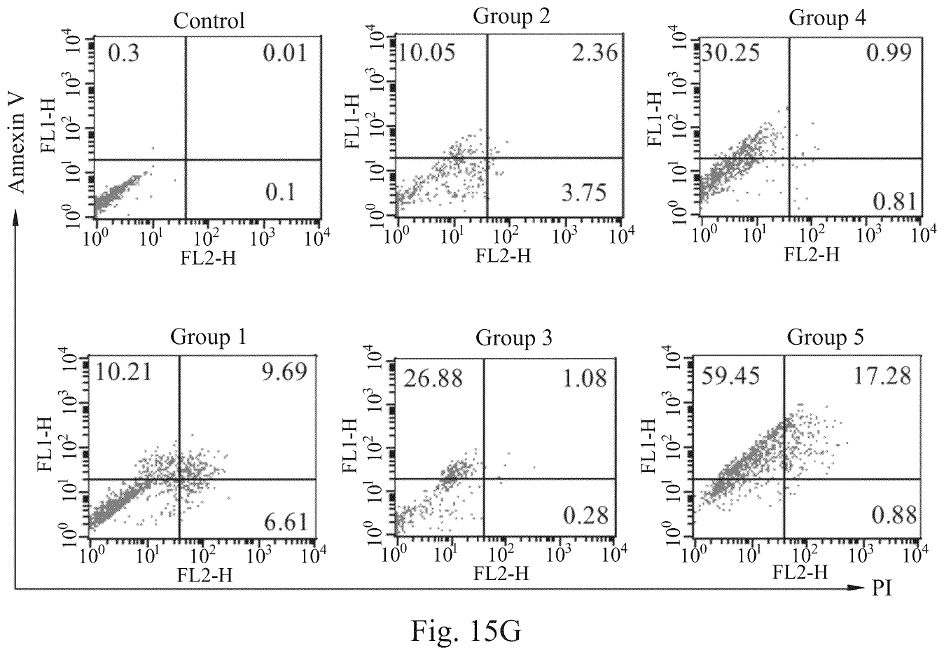

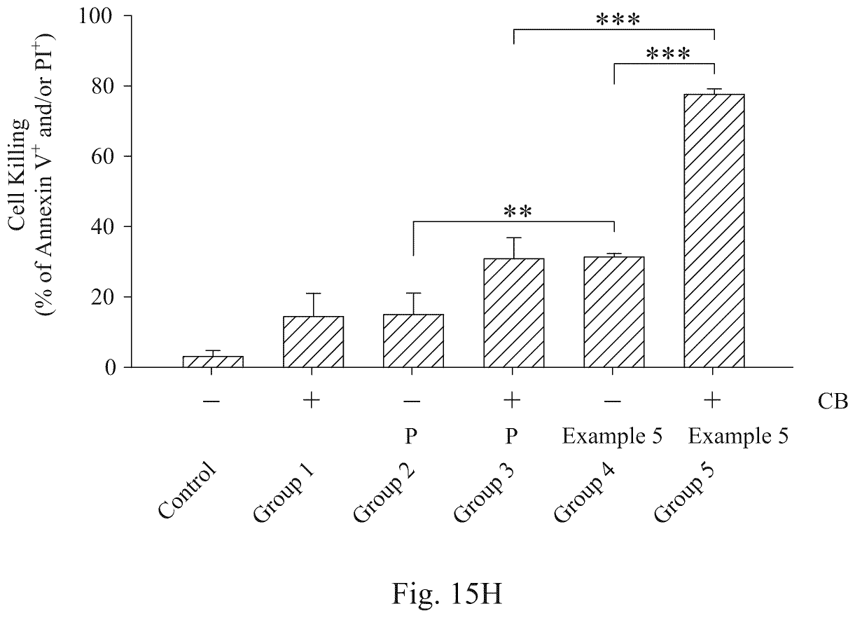

Reference is made to FIG. 15A, FIG. 15B, FIG. 15C, FIG. 15D, FIG. 15E, FIG. 15F, FIG. 15G, FIG. 15H and Table 5, which show analytical results of tumor cell death induced by the BAG6 specific chimeric antigen receptor expressing cells according to Example 5 of the present disclosure, wherein “P” represents parental primary T cells in FIG. 15B, FIG. 15D, FIG. 15F and FIG. 15H. FIG. 15A is a graph showing the analytical results of the human breast cancer cell line MDA-MB-231, and FIG. 15B is a statistical chart of FIG. 15A after three independent trials, wherein “Dox” represents doxorubicin. FIG. 15C is a graph showing the analytical results of the human malignant brain tumor cell line DBTRG, and FIG. 15D is a statistical chart of FIG. 15C after three independent trials, wherein “TMZ” represents temozolomide. FIG. 15E is a graph showing the analytical results of the human pancreatic cancer cell line AsPC-1, and FIG. 15F is a statistical chart of FIG. 15E after three independent trials, wherein “Gem” represents gemcitabine. FIG. 15G is a graph showing the analytical results of the human ovarian cancer cell line SKOV3, and FIG. 15H is a statistical chart of FIG. 15G after three independent trials, wherein “CB” represents carboplatin. Table 5 shows the death rate of each group.

TABLE 5
Analytical results of Example 5

| | Control | Group 1 | Group 2 | Group 3 | Group 4 | Group 5 |
|---|---|---|---|---|---|---|
| MDA- | 3.6725 ± | 6.5250 ± | 9.9900 ± | 20.1200 ± | 37.5600 ± | 53.6000 ± |
| MB-231 | 1.2181 | 1.2487 | 4.3398 | 8.9176 | 2.5729 | 0.8061 |
| DBTRG | 1.5200 ± | 5.5450 ± | 15.9600 ± | 23.6950 ± | 40.9100 ± | 64.0200 ± |
| | 0.4205 | 2.8119 | 2.1332 | 3.5835 | 6.3781 | 2.4324 |
| AsPC-1 | 5.2400 ± | 19.1850 ± | 26.5600 ± | 36.0750 ± | 41.3067 ± | 63.7133 ± |
| | 0.8421 | 3.4624 | 1.6037 | 2.6799 | 3.3421 | 1.3213 |
| SKOV3 | 6.2183 ± | 10.8100 ± | 16.5060 ± | 26.0840 ± | 40.1733 ± | 75.0525 ± |
| | 5.0170 | 4.3249 | 4.6054 | 4.6600 | 5.9923 | 2.4108 |

The results of Table 5, FIG. 15A and FIG. 15B show that in the human breast cancer cell line MDA-MB-231, the death rate of the human breast cancer cell line MDA-MB-231 in the group 4 treated with Example 5 was approximately 37.6%, and there was a statistically significant difference (p<0.001) compared to the group 2. In addition, the death rate of the human breast cancer cell line MDA-MB-231 in the group 5 treated with Example 5 and doxorubicin was over 50%, and there was a statistically significant difference (p<0.01) compared to the group 4 and a statistically significant difference (p<0.001) compared to the group 3, respectively.

The results of Table 5, FIG. 15C and FIG. 15D show that in the human malignant brain tumor cell line DBTRG, the death rate of the human malignant brain tumor cell line DBTRG in the group 4 treated with Example 5 was about 40.9%, and there was a statistically significant difference (p<0.01) compared to the group 2. In addition, the death rate of the human malignant brain tumor cell line DBTRG in the group 5 treated with Example 5 and temozolomide was over 60%, and there was a statistically significant difference (p<0.01) compared to the group 4 and a statistically significant difference (p<0.001) compared to the group 3, respectively.

The results of Table 5, FIG. 15E and FIG. 15F show that in the human pancreatic cancer cell line AsPC-1, the death rate of the human pancreatic cancer cell line AsPC-1 in the group 4 treated with Example 5 was about 41.3%, and there was a statistically significant difference (p<0.05) compared to the group 2. In addition, the death rate of the human pancreatic cancer cell line AsPC-1 in the group 5 treated with Example 5 and gemcitabine could reach about 60%, and there was a statistically significant difference (p<0.05) compared to the group 4 and a statistically significant difference (p<0.01) compared to the group 3, respectively.

The results of Table 5, FIG. 15G and FIG. 15H show that in the human ovarian cancer cell line SKOV3, the death rate of the human ovarian cancer cell line SKOV3 in the group 4 treated with Example 5 was about 40.2%, and there was a statistically significant difference (p<0.01) compared to the group 2. The death rate of the human ovarian cancer cell line SKOV3 in the group 5 treated with Example 5 and carboplatin could reach 75.1%, and there was a statistically significant difference (p<0.001) compared to the group 4 and a statistically significant difference (p<0.001) compared to the group 3, respectively.

### 4.2. Example 6

The BAG6 specific chimeric antigen receptor of 3rd embodiment was transduced into the primary NK cell to obtain a BAG6 specific chimeric antigen receptor expressing cell of Example 6 of the present disclosure (hereinafter referred to as Example 6). The effects of Example 6 and the pharmaceutical composition for treating cancer including Example 6 of the present disclosure on inducing the death of the breast cancer cells, the glioblastoma multiforme cells, the pancreatic cancer cells, and the ovarian cancer cells were further demonstrated in following experiments.

First, the human breast cancer cell line MDA-MB-231, the human malignant brain tumor cell line DBTRG, the human pancreatic cancer cell line AsPC-1 and the human ovarian cancer cell line SKOV3 were seeded in a 12-well plate at a density of 1×10⁵ cells/well. The cells were subsequently incubated for 48 hours. Each type of the tumor cells was divided into six groups. In a control, the tumor cells were untreated. In a group 1, the tumor cells were treated with the chemotherapy drug. In a group 2, the tumor cells were treated with the parental primary NK cell. In a group 3, the tumor cells were treated with the parental primary NK cell and the chemotherapy drug. In a group 4, the tumor cells were treated with Example 6. In a group 5, the tumor cells were treated with Example 6 and the chemotherapy drug. The chemotherapy drug used for treating the human breast cancer cell line MDA-MB-231 was doxorubicin (200 nM), the chemotherapy drug used for treating the human malignant brain tumor cell line DBTRG was temozolomide (80 μg/mL), the chemotherapy drug used for treating the human pancreatic cancer cell line AsPC-1 was gemcitabine (20 μM), and the chemotherapy drug used for treating the human ovarian cancer cell line SKOV3 was carboplatin (20 μM). In the group 4 and the group 5, the number of Example 6 treated was 1×10⁵ cells. In the group 2 and the group 3, the number of the parental primary NK cell treated was 1×10⁵ cells. The treated cells of each group were stained with Annexin V-FITC and PI, and the apoptosis and the death of the tumor cells were detected by the flow cytometry. The sum of the percentage of cells stained with Annexin V-FITC and/or PI were calculated to obtain the cytotoxicity. The results of the cytotoxicity were counted after three independent trials in each group.

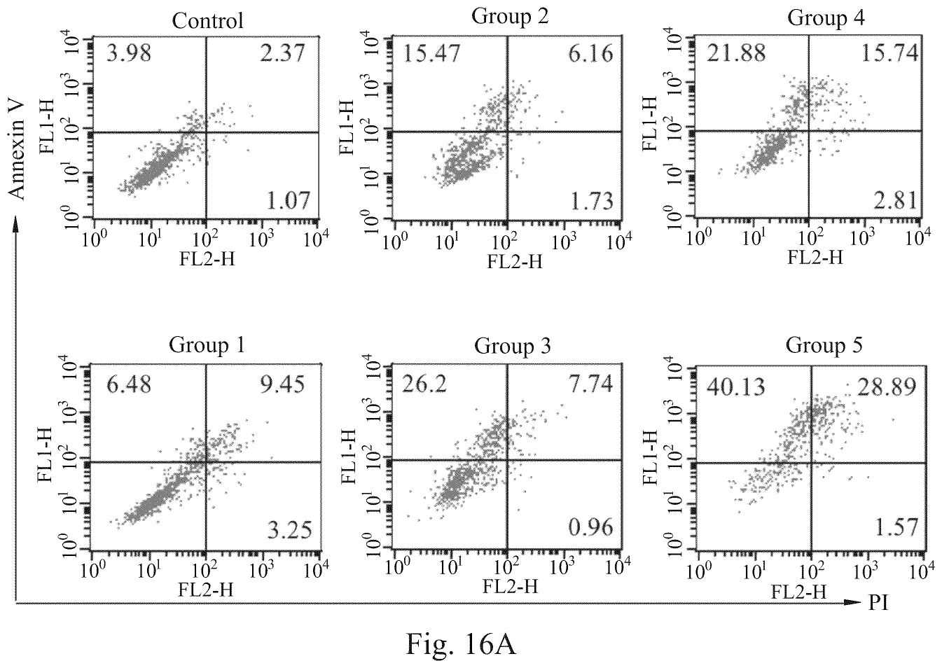

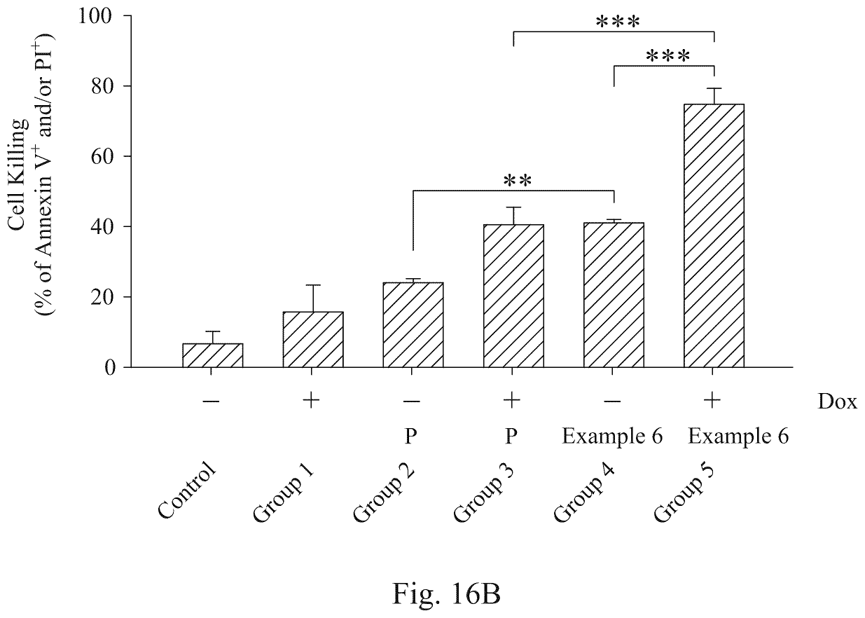

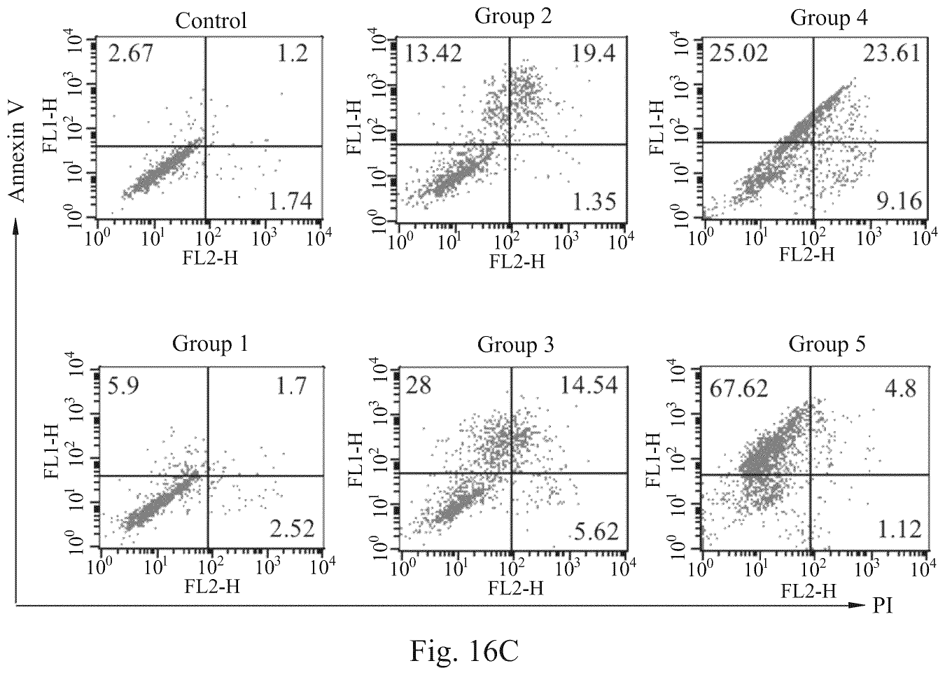

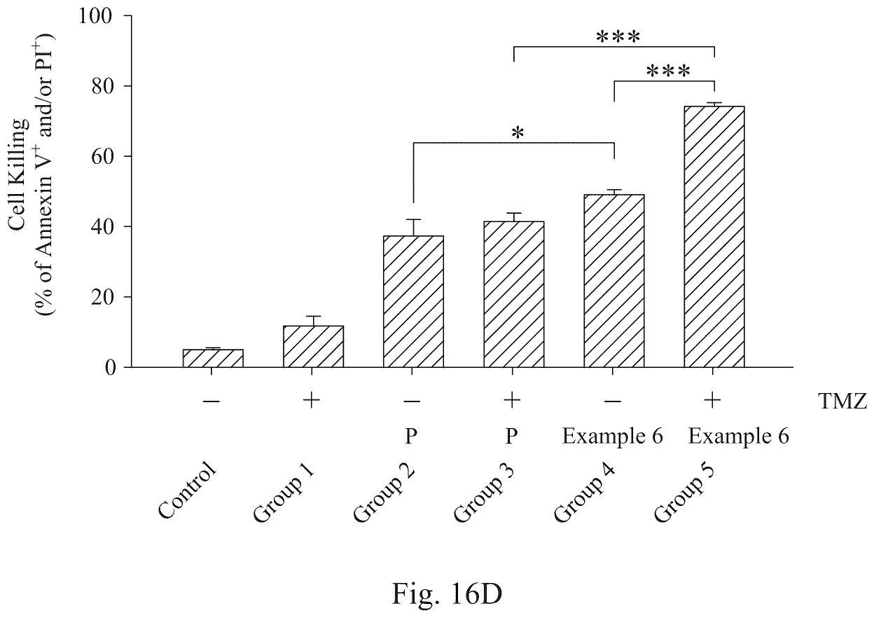

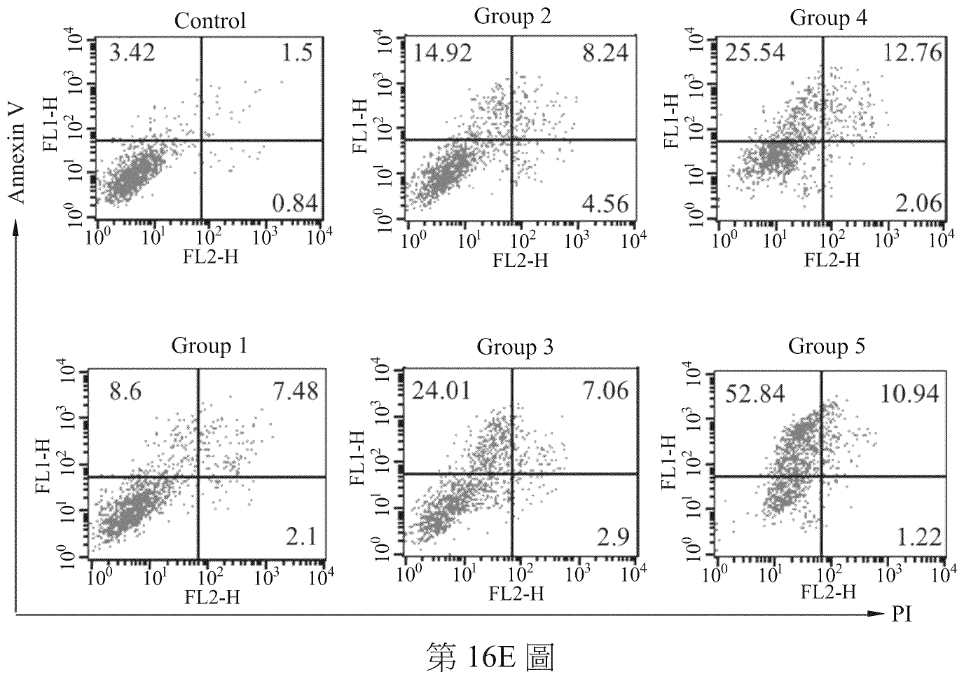

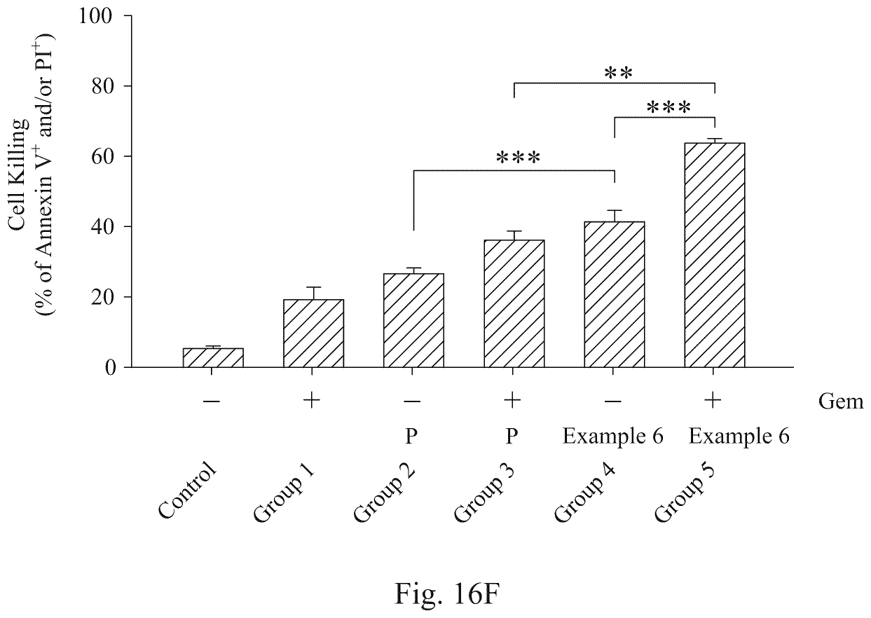

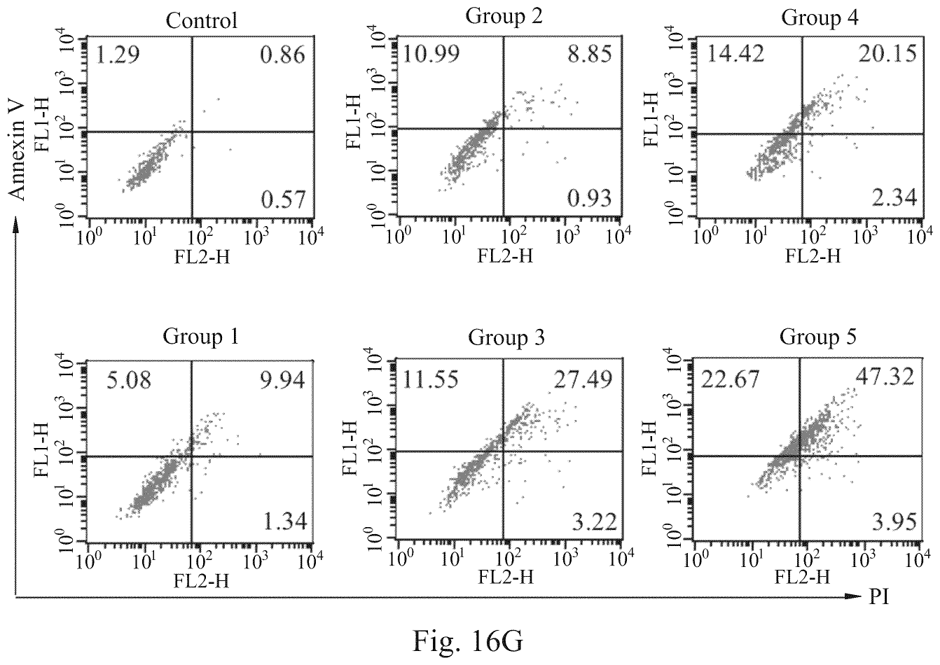

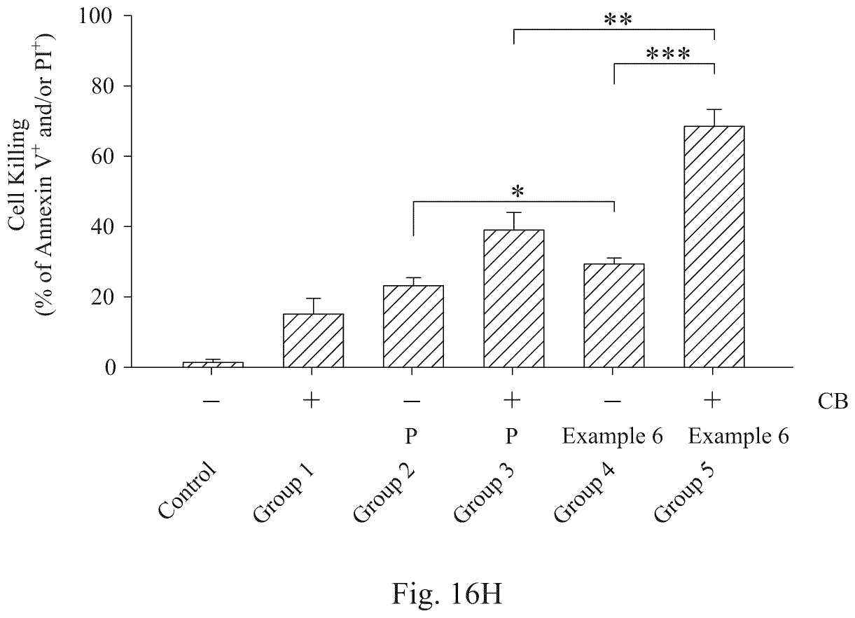

Reference is made to FIG. 16A, FIG. 16B, FIG. 16C, FIG. 16D, FIG. 16E, FIG. 16F, FIG. 16G, FIG. 16H and Table 6, which show analytical results of tumor cell death induced by the BAG6 specific chimeric antigen receptor expressing cells according to Example 6 of the present disclosure, wherein “P” represents parental primary NK cell in FIG. 16B, FIG. 16D, FIG. 16F and FIG. 16H. FIG. 16A is a graph showing the analytical results of the human breast cancer cell line MDA-MB-231, and FIG. 16B is a statistical chart of FIG. 16A after three independent trials, wherein “Dox” represents doxorubicin. FIG. 16C is a graph showing the analytical results of the human malignant brain tumor cell line DBTRG, and FIG. 16D is a statistical chart of FIG. 16C after three independent trials, wherein “TMZ” represents temozolomide. FIG. 16E is a graph showing the analytical results of the human pancreatic cancer cell line AsPC-1, and FIG. 16F is a statistical chart of FIG. 16E after three independent trials, wherein “Gem” represents gemcitabine. FIG. 16G is a graph showing the analytical results of the human ovarian cancer cell line SKOV3, and FIG. 16H is a statistical chart of FIG. 16G after three independent trials, wherein “CB” represents carboplatin. Table 6 shows the death rate of each group.

TABLE 6
Analytical results of Example 6

| | Control | Group 1 | Group 2 | Group 3 | Group 4 | Group 5 |
|---|---|---|---|---|---|---|
| MDA- | 6.6533 ± | 15.7033 ± | 24.0367 ± | 40.5367 ± | 41.0950 ± | 74.7167 ± |
| MB-231 | 3.5035 | 7.6338 | 1.1035 | 4.9684 | 0.9405 | 4.5400 |
| DBTRG | 5.0150 ± | 13.9750 ± | 34.5800 ± | 43.3533 ± | 66.3250 ± | 83.8000 ± |
| | 0.4409 | 4.9209 | 0.5798 | 3.7230 | 8.4641 | 1.5427 |
| AsPC-1 | 5.2400 ± | 19.1850 ± | 26.5600 ± | 36.0750 ± | 41.3067 ± | 63.7133 ± |
| | 0.8421 | 3.4624 | 1.6037 | 2.6799 | 3.3421 | 1.3213 |
| SKOV3 | 1.3875 ± | 15.0975 ± | 23.1733 ± | 38.9700 ± | 29.3400 ± | 68.5067 ± |
| | 0.8887 | 4.4817 | 2.2817 | 4.9930 | 1.6829 | 4.7110 |

The results of Table 6, FIG. 16A and FIG. 16B show that in the human breast cancer cell line MDA-MB-231, the death rate of the human breast cancer cell line MDA-MB-231 in the group 4 treated with Example 6 was approximately 41.1%, and there was a statistically significant difference (p<0.01) compared to the group 2. In addition, the death rate of the human breast cancer cell line MDA-MB-231 in the group 5 treated with Example 6 and doxorubicin could reach 74.7%, and there was a statistically significant difference (p<0.001) compared to the group 4 and a statistically significant difference (p<0.001) compared to the group 3, respectively.

The results of Table 6, FIG. 16C and FIG. 16D show that in the human malignant brain tumor cell line DBTRG, the death rate of the human malignant brain tumor cell line DBTRG in the group 4 treated with Example 6 was 66.3%, and there was a statistically significant difference (p<0.05) compared to the group 2. In addition, the death rate of the human malignant brain tumor cell line DBTRG in the group 5 treated with Example 6 and temozolomide was close to 83.8%, and there was a statistically significant difference (p<0.001) compared to the group 4 and a statistically significant difference (p<0.001) compared to the group 3, respectively.

The results of Table 6, FIG. 16E and FIG. 16F show that in the human pancreatic cancer cell line AsPC-1, the death rate of the human pancreatic cancer cell line AsPC-1 in the group 4 treated with Example 6 was about 41.3%, and there was a statistically significant difference (p<0.001) compared to the group 2. In addition, the death rate of the human pancreatic cancer cell line AsPC-1 in the group 5 treated with Example 6 and gemcitabine was about 63.7%, and there was a statistically significant difference (p<0.001) compared to the group 4 and a statistically significant difference (p<0.01) compared to the group 3, respectively.

The results of Table 6, FIG. 16G and FIG. 16H show that in the human ovarian cancer cell line SKOV3, the death rate of the human ovarian cancer cell line SKOV3 in the group 4 treated with Example 6 was about 29.3%, and there was a statistically significant difference (p<0.05) compared to the group 2. In addition, the death rate of the human ovarian cancer cell line SKOV3 in the group 5 treated with Example 6 and carboplatin could reach 68.5%, and there was a statistically significant difference (p<0.001) compared to the group 4 and a statistically significant difference (p<0.01) compared to the group 3, respectively.

The results of Table 5, Table 6 and FIG. 15A to FIG. 16H show that the BAG6 specific chimeric antigen receptor expressing cell of 3rd embodiment can be used to treat with the breast cancer cell, the glioblastoma multiforme cell, the pancreatic cancer cell or the ovarian cancer cell for excellent cell killing. Therefore, the BAG6 specific chimeric antigen receptor expressing cell of the present disclosure can be used for inhibiting the proliferation of the tumor cells in the subject in need for the treatment of the tumor. In addition, the results of FIG. 2A, FIG. 2B and FIG. 4 show that treatment with the chemotherapy drug increased the expression of BAG6 and the expression of B7-H6 on the cell membrane of the tumor cells, and the BAG6 specific chimeric antigen receptor expressed by the BAG6 specific chimeric antigen receptor expressing cell of 3rd embodiment can specifically bind to BAG6 and B7-H6. Therefore, the treatment of treating the chemotherapy drug first and then treating the BAG6 specific chimeric antigen receptor expressing cell of 3rd embodiment, or the simultaneous treatment of the chemotherapy drug and the BAG6 specific chimeric antigen receptor expressing cell of 3rd embodiment can achieve a better effect on killing the tumor cells. The results indicate that the pharmaceutical composition for treating cancer of the present disclosure can effectively inhibit the growth of the tumor cells and treat cancer. Preferably, the pharmaceutical composition for treating cancer can include the chemotherapy drug.

To sum up, the BAG6 specific chimeric antigen receptor of the present disclosure has excellent specific binding ability to the tumor cells, in particular, specific binding to BAG6 expressed on the cell membrane of the tumor cells. Accordingly, the BAG6 specific chimeric antigen receptor expressing cell of the present disclosure, which expresses the BAG6 specific chimeric antigen receptor of the present disclosure, can specifically target the tumor cells to avoid the off-target effect, thereby effectively killing the tumor cells. Therefore, the BAG6 specific chimeric antigen receptor expressing cell can be used for inhibiting the proliferation of the tumor cells in the subject in need for the treatment of the tumor. The pharmaceutical composition for treating cancer includes the BAG6 specific chimeric antigen receptor expressing cell of the present disclosure and the pharmaceutically acceptable carrier, which can effectively kill the tumor cells and thereby treat cancer. The pharmaceutical composition for treating cancer further including the chemotherapy drug can increase the BAG6 expression level on the plasma membrane of the tumor cells, thereby enhancing the killing effect of the BAG6 specific chimeric antigen receptor expressing cell on the tumor cells. Accordingly, the pharmaceutical composition for treating cancer further including the chemotherapy drug has more excellent tumor cell toxicity.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A BAG6 specific chimeric antigen receptor specific to BCL2 associated athanogene 6 (BAG6) comprising, in order from an N-terminus to a C-terminus:
a BAG6 antigen recognition domain, a transmembrane domain and a cytoplasmic domain;
wherein the BAG6 antigen recognition domain comprises a monoclonal antibody fragment specific to BAG6, and the BAG6 antigen recognition domain comprises the amino acid sequence of SEQ ID NO: 1.

2. The BAG6 specific chimeric antigen receptor of claim 1, further comprising a HLA-G antigen recognition domain comprising the amino acid sequence of SEQ ID NO: 2, wherein the HLA-G antigen recognition domain is linked to the C-terminus of the BAG6 antigen recognition domain, and the HLA-G antigen recognition domain comprises a monoclonal antibody fragment specific to human leukocyte antigen G (HLA-G).

3. The BAG6 specific chimeric antigen receptor of claim 1, further comprising a B7-H6 antigen recognition domain comprising the amino acid sequence of SEQ ID NO: 3, wherein the B7-H6 antigen recognition domain is linked to the C-terminus of the BAG6 antigen recognition domain, and the B7-H6 antigen recognition domain comprises a monoclonal antibody fragment specific to B7 homolog 6 (B7-H6).

4. The BAG6 specific chimeric antigen receptor of claim 1, wherein the transmembrane domain is a CD28 transmembrane domain comprising the amino acid sequence of SEQ ID NO: 4, a CD8 transmembrane domain comprising the amino acid sequence of SEQ ID NO: 5 or a KIR transmembrane domain comprising the amino acid sequence of SEQ ID NO: 6.

5. The BAG6 specific chimeric antigen receptor of claim 1, wherein the cytoplasmic domain is a IL2 receptor β chain signaling domain comprising the amino acid sequence of SEQ ID NO: 7, a modified CD3ζ signaling domain comprising the amino acid sequence of SEQ ID NO: 8, a DAP12 signaling domain comprising the amino acid sequence of SEQ ID NO: 9, a CD3ζ signaling domain comprising the amino acid sequence of SEQ ID NO: 10, a 4-1BB signaling domain comprising the amino acid sequence of SEQ ID NO: 11 or a combination thereof.

6. The BAG6 specific chimeric antigen receptor of claim 1, further comprising a suicide protein comprising the amino acid sequence of SEQ ID NO: 12, wherein the suicide protein is linked to the C-terminus of the cytoplasmic domain.

7. A nucleic acid encoding the BAG6 specific chimeric antigen receptor of claim 1, comprising, in order from a 5′ end to a 3′ end:
a BAG6 antigen recognition domain coding fragment comprising the nucleic acid sequence of SEQ ID NO: 13, a transmembrane domain coding fragment and a cytoplasmic domain coding fragment.

8. The nucleic acid of claim 7, further comprising a HLA-G antigen recognition domain coding fragment comprising the nucleic acid sequence of SEQ ID NO: 14, wherein the HLA-G antigen recognition domain coding fragment is linked to the 3′ end of the BAG6 antigen recognition domain coding fragment.

9. The nucleic acid of claim 7, further comprising a B7-H6 antigen recognition domain coding fragment comprising the nucleic acid sequence of SEQ ID NO: 15, wherein the B7-H6 antigen recognition domain is linked to the 3′ end of the BAG6 antigen recognition domain coding fragment.

10. The nucleic acid of claim 7, wherein the transmembrane domain coding fragment is a CD28 transmembrane domain coding fragment comprising the nucleic acid sequence of SEQ ID NO: 16, a CD8 transmembrane domain coding fragment comprising the nucleic acid sequence of SEQ ID NO: 17 or a KIR transmembrane domain coding fragment comprising the nucleic acid sequence of SEQ ID NO: 18.

11. The nucleic acid of claim 7, wherein the cytoplasmic domain coding fragment is a IL2 receptor β chain signaling domain coding fragment comprising the nucleic acid sequence of SEQ ID NO: 19, a modified CD3ζ signaling domain coding fragment comprising the nucleic acid sequence of SEQ ID NO: 20, a DAP12 signaling domain coding fragment comprising the nucleic acid sequence of SEQ ID NO: 21, a CD3ζ signaling domain coding fragment comprising the nucleic acid sequence of SEQ ID NO: 22, a 4-1BB signaling domain coding fragment comprising the nucleic acid sequence of SEQ ID NO: 23 or a combination thereof.

12. The nucleic acid of claim 7, further comprising a suicide gene comprising the nucleic acid sequence of SEQ ID NO: 24, wherein the suicide gene is linked to the 3′ end of the cytoplasmic domain coding fragment.

13. A BAG6 specific chimeric antigen receptor expression plasmid comprising, in order from a 5′ end to a 3′ end:
a promoter comprising the nucleic acid sequence of SEQ ID NO: 25; and
the nucleic acid of claim 7.

14. The BAG6 specific chimeric antigen receptor expression plasmid of claim 13, further comprising a suicide gene comprising the nucleic acid sequence of SEQ ID NO: 24, wherein the suicide gene is linked to the 3′ end of the nucleic acid.

15. A BAG6 specific chimeric antigen receptor expressing cell, comprising:
an immune cell; and
the BAG6 specific chimeric antigen receptor expression plasmid of claim 13.

16. The BAG6 specific chimeric antigen receptor expressing cell of the claim 15, wherein the immune cell is a T lymphocyte or a natural killer (NK) cell.

17. A pharmaceutical composition for treating cancer, comprising:
the BAG6 specific chimeric antigen receptor expressing cell of claim 15; and
a pharmaceutically acceptable carrier.

18. The pharmaceutical composition for treating cancer of the claim 17, further comprising a chemotherapy drug.

19. The pharmaceutical composition for treating cancer of the claim 18, wherein the chemotherapy drug is doxorubicin, temozolomide, gemcitabine or carboplatin.

20. A method for inhibiting a proliferation of a tumor cell comprising administering a composition comprising a plurality of the BAG6 specific chimeric antigen receptor expressing cells of claim 15 to a subject in need for a treatment of a tumor.

21. The method of claim 20, wherein the tumor cell is a breast cancer cell, a glioblastoma multiforme cell, a pancreatic cancer cell or an ovarian cancer cell.


SEQUENCE LISTING (25 sequences)

SEQ ID NO: 1 - protein, 239 aa, synthetic construct
EVQLLESGGGLVQPGGSLRLSCAASGFTFSSYAMSWVRQAPGKGLEWVSSIAPKGGKTAYADSVKGRFTISRDNSKNTLYLQMNSLRAEDTAVYYCAKNLATFDYWGQGTLVTVSSGGGGSGGGGSGGGGSTDIQMTQSPSSLSASVGDRVTITCRASQSISSYLNWYQQKPGKAPKLLIYSASTLQSGVPSRFSGSGSGTDFTLTISSLQPEDFATYYCQQAEQWPPTFGQGTKVEIK

SEQ ID NO: 2 - protein, 246 aa, synthetic construct
EVQLQESGGGLVQPKGSLKLSCAAFGFTFNTYAMHWVRQAPGKGLEWVARIRSKSNNYATYYADSVKDRFTISRDDSQSMLSLQMNNLKTEDTAIYYCVRGGYWSFDVWGAGTTVTVSSGGGGSGGGGSGGGGSDIVITQTTPSVPVTPGESVSISCRSSKSLLHSNGNTYLYWFLQRPGQSPQLLISRMSSLASGVPDRFSGSGSGTAFTLRISRVEAEDVGVYYCMQHLEYPYTFGGGTKLEIK

SEQ ID NO: 3 - protein, 252 aa, synthetic construct
DIKMTQSPSSMYASLGERVTITCKASQDINSYLSWFQQKPGKSPKTLCDRIYRANRLVDGVPSRFSGSGSGQDYSLTISSLEYEDMGIYYCLQYDEFCDRCDRPYTFGGGTKLEIKGGGGSGGGGSGGGGSQVQLQQSGAELMKPGASVKLSCKATGYTFTGYWIEWIKQRPGHGLEWCDRIGEILPGTGSTNYNEKFKGKATFTADTSSNTAYMQLSSLTTEDSAIYCDRYCAIPGPMDYWGQGTSVTVSS

SEQ ID NO: 4 - protein, 68 aa, synthetic construct
FWVLVVVGGVLACYSLLVTVAFIIFWVRSKRSRLLHSDYMNMTPRRPGPTRKHYQPYAPPRDFAAYRS

SEQ ID NO: 5 - protein, 55 aa, synthetic construct
IASQPLSLRPEACRPAAGGAVHTRGLDFACDIYIWAPLAGTCGVLLLSLVITLYC

SEQ ID NO: 6 - protein, 75 aa, synthetic construct
SPTEPSSKTGNPRHLHVLIGTSVVKIPFTILLFFLLHRWCSNKKNAAVMDQEPAGNRTVNSEDSDEQDHQEVSYA

SEQ ID NO: 7 - protein, 94 aa, synthetic construct
NCRNTGPWLKKVLKCNTPDPSKFFSQLSSEHGGDVQKWLSSPFPSSSFSPGGLAPEISPLEVLERDKVTQLLPLNTDAYLSLQELQGQDPTHLV

SEQ ID NO: 8 - protein, 112 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDAYRHQALPP

SEQ ID NO: 9 - protein, 113 aa, synthetic construct
MGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 10 - protein, 111 aa, synthetic construct
RVKFSRSADAPAYQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 42 aa, synthetic construct
KRGRKKLLYIFKQPFMRPVQTTQEEDGCSCRFPEEEEGGCEL

SEQ ID NO: 12 - protein, 439 aa, synthetic construct
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSTNRQAAKLSKPTLENLTPVVLRPEIRKPEVLRPETPRPVDIGSGGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 13 - DNA, 717 bp, synthetic construct
  source 1..717: mol_type = other DNA
gaagtgcagctgctggaatctggcggaggactggttcaacctggcggctctctgagactgtcttgtgccgccagcggcttcacctttagcagctacgccatgagctgggtccgacaggctcctggcaaaggccttgaatgggtgtcctctatcgcccctaaaggcggcaagacagcctacgccgatagcgtgaagggcagattcaccatcagccgggacaacagcaagaacaccctgtacctgcagatgaacagcctgagagccgaggacaccgccgtgtactactgcgccaaaaacctggccaccttcgactattggggccagggcacactggtcacagtttctagcggaggcggaggttctggcggcggaggaagtggtggcggaggctctacagacatccagatgacacagagccccagcagcctgtctgccagcgtgggagatagagtgaccatcacctgtagggccagccagagcatcagcagctacctgaactggtatcagcagaagcccggcaaggcccctaaactgctgatctacagcgccagcacactgcagagcggagtgcctagcagattttctggcagcggctccggcaccgatttcaccctgaccatatctagcctgcagcctgaggacttcgccacctactattgtcagcaggccgagcagtggcctcctacatttggccagggaacaaaggtggaaatcaag

SEQ ID NO: 14 - DNA, 738 bp, synthetic construct
  source 1..738: mol_type = other DNA
gaggttcagctgcaagagtctggcggaggactggtgcagcctaagggaagcctgaagctgagctgtgccgccttcggcttcaccttcaacacctacgccatgcactgggtccgacaggcccctggaaaaggccttgaatgggtcgcccggatcagaagcaagagcaacaattacgccacctactacgccgacagcgtgaaggacagattcaccatcagccgggacgacagccagagcatgctgagcctgcagatgaacaacctgaaaaccgaggacaccgccatctactactgcgtcagaggcggctactggtccttcgatgtttggggagccggcaccaccgtgacagtttctagcggaggcggtggatctggcggcggaggaagtggtggcggaggttctgatatcgtgatcacccagaccacacctagcgtgccagtgacacctggcgagagcgtgtccatcagctgcagaagcagcaagagcctgctgcacagcaacggcaatacctacctgtactggttcctgcagaggcccggacagtctcctcagctgctgatctccagaatgagcagcctggctagcggcgtgcccgatagattttctggcagcggctctggcaccgccttcacactgagaatcagcagagtggaagccgaggacgtgggcgtgtactactgtatgcagcacctggaatacccctacaccttcggcggaggcaccaagctggaaatcaag

SEQ ID NO: 15 - DNA, 711 bp, synthetic construct
  source 1..711: mol_type = other DNA
gacatcaagatgacacagagccccagctctatgtacgccagcctgggagagagagtgaccattacctgcaaggccagccaggacatcaacagctacctgagctggttccagcagaagcccggcaagagccctaagacactgtgcgacagaatctaccgggccaacagactggtggatggcgtgccaagcagattttctggcagcggcagcggccaggattacagcctgacaatcagcagcctggaatacgaggacatgggcatctactactgcctgcagtacgacgagttctgcgacagatgcgacaggccctacacatttggcggaggcaccaagctggaaatcaagcaggttcagctgcagcagtctggcgccgaacttatgaagcctggcgcctctgtgaagctgagctgtaaagccaccggctacacctttaccggctactggatcgagtggatcaagcagaggcctggccatggactcgagtggtgcgatagaatcggcgagatcctgcctggcaccggcagcaccaactacaacgagaagttcaagggcaaagccaccttcaccgccgacaccagcagcaacacagcctacatgcagctgagcagcctgaccacagaggacagcgccatctactgcgaccggtactgtgccattcctggacctatggactattggggccagggcacaagcgtgaccgttagctct

SEQ ID NO: 16 - DNA, 204 bp, synthetic construct
  source 1..204: mol_type = other DNA
ttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 17 - DNA, 72 bp, synthetic construct
  source 1..72: mol_type = other DNA
atctacatttgggcccctctggctggtacttgcggggtcctgctgctttcactcgtgatcactctttactgt

SEQ ID NO: 18 - DNA, 225 bp, synthetic construct
  source 1..225: mol_type = other DNA
tcacccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgca

SEQ ID NO: 19 - DNA, 282 bp, synthetic construct
  source 1..282: mol_type = other DNA
aactgcaggaacaccgggccatggctgaagaaggtcctgaagtgtaacaccccagacccctcgaagttcttttcccagctgagctcagagcatggaggagacgtccagaagtggctctcttcgcccttcccctcatcgtccttcagccctggcggcctggcacctgagatctcgccactagaagtgctggagagggacaaggtgacgcagctgctccccctgaacactgatgcctacttgtccctccaagaactccagggtcaggacccaactcacttggtg

SEQ ID NO: 20 - DNA, 339 bp, synthetic construct
  source 1..339: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcctatcgccaccaggccctgcccccttaa

SEQ ID NO: 21 - DNA, 339 bp, synthetic construct
  source 1..339: mol_type = other DNA
atggggggacttgaaccctgcagcaggctcctgctcctgcctctcctgctggctgtaagtggtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaa

SEQ ID NO: 22 - DNA, 336 bp, synthetic construct
  source 1..336: mol_type = other DNA
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 23 - DNA, 126 bp, synthetic construct
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 24 - DNA, 1317 bp, synthetic construct
  source 1..1317: mol_type = other DNA
atgggagtgcaggtggaaaccatctccccaggagacgggcgcaccttccccaagcgcggccagacctgcgtggtgcactacaccgggatgcttgaagatggaaagaaagtggattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggaggaggttctactaacaggcaagcagcaaagttgtcgaagccaaccctagaaaaccttaccccagtggtgctcagaccagagattcgcaaaccagaggttctcagaccggaaacacccagaccagtggacattggttctggaggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcagcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatca

SEQ ID NO: 25 - DNA, 1335 bp, synthetic construct
  source 1..1335: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga